(19) United States
(12) Patent Application Publication
(10) Pub. No.: US 2016/0108038 A1
(43) Pub. Date: Apr. 21, 2016

(54) BICYCLIC ARYL SULFIDE AND ARYL SULFOXIDE DERIVATIVES AS PEST CONTROL AGENT

(71) Applicant: BAYER CROPSCIENCE AKTIENGESELLSCHAFT, Monheim am Rhein (DE)

(72) Inventors: Bernd ALIG, Koenigswinter (DE); Silvia CEREZO-GALVEZ, Langenfeld (DE); Reiner FISCHER, Monheim (DE); Adeline KOEHLER, Langenfeld (DE); Julia Johanna HAHN, Duesseldorf (DE); Kerstin ILG, Koeln (DE); Daniela PORTZ, Vettweiss (DE); Olga MALSAM, Roesrath (DE); Peter LOESEL, Leverkusen (DE); Angela BECKER, Duesseldorf (DE); Ulrich GOERGENS, Ratingen (DE)

(21) Appl. No.: 14/895,326

(22) PCT Filed: May 30, 2014
(86) PCT No.: PCT/EP2014/061231

§ 371 Date: Dec. 2, 2015

(30) Foreign Application Priority Data
Jun. 4, 2013 (EP) 13170445.4

(51) Int. Cl. C07D 471/04; A01N 43/90

(52) U.S. Cl. CPC C07D 471/04; A01N 43/90

(57) ABSTRACT

The present application relates to novel heterocyclic compounds, to processes for their preparation, to their use for controlling animal pests including arthropods and in particular insects and acarids, and to intermediates for preparing the heterocyclic compounds.

The present application relates to novel heterocyclic compounds, to processes for their preparation, to their use for controlling animal pests including arthropods and in particular insects and to intermediates for preparing the heterocyclic compounds.

Arylquinazolinones and their insecticidal and acaricidal action are already known from WO 2010/100189 A1.

Crop protection compositions, which also include pesticides, have to meet many demands, for example in relation to efficacy, persistence, and spectrum of their action and possible use. Questions of toxicity and of combinability with other active compounds or formulation auxiliaries play a role, as does the question of the expense that the synthesis of an active compound requires. In addition, resistances can occur. For all these reasons, the search for novel crop protection agents cannot be considered to be complete, and there is a constant need for novel compounds having properties which, compared to the known compounds, are improved at least in relation to individual aspects.

It was an object of the present invention to provide compounds which widen the spectrum of the pesticides in various aspects.

This object, and further objects which are not stated explicitly but can be discerned or derived from the connections discussed herein, are achieved by novel compounds of the formula (I)

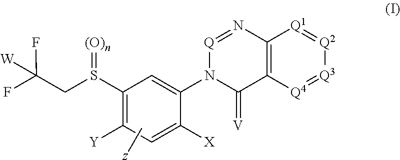

in which
V represents oxygen, sulfur or substituted nitrogen;
Q represents substituted carbon or represents nitrogen;

Q¹, Q², Q³, Q⁴ each independently of one another represent substituted carbon or represent nitrogen, where at least one of the structural elements Q¹, Q², Q³, Q⁴ is nitrogen and at most two of the structural elements Q¹, Q², Q³, Q⁴ are nitrogen;
W represents hydrogen or halogen;
n represents the number 0, 1 or 2;
X, Y and z each independently of one another
   represent hydrogen, halogen, hydroxyl, amino, cyano, nitro, OCN, SCN, SF₅; or
   represent trialkylsilyl, alkyl, haloalkyl, cyanoalkyl, hydroxyalkyl, alkoxycarbonylalkyl, alkoxyalkyl, alkenyl, haloalkenyl, cyanoalkenyl, alkynyl, haloalkynyl, cyanoalkynyl, alkoxy, haloalkoxy, cyanoalkoxy, hydroxycarbonylalkoxy, alkoxycarbonylalkoxy, alkoxyalkoxy, alkylhydroxyimino, alkoxyimino, alkylalkoxyimino, haloalkylalkoxyimino, alkylthio, haloalkylthio, alkoxyalkylthio, alkylthioalkyl, alkylsulfinyl, haloalkylsulfinyl, alkoxyalkylsulfinyl, alkylsulfinylalkyl, alkylsulfonyl, haloalkylsulfonyl, alkoxyalkylsulfonyl, alkylsulfonylalkyl, alkylsulfonyloxy, alkylcarbonyl, haloalkylcarbonyl, carboxyl, alkylcarbonyloxy, alkoxycarbonyl, haloalkoxycarbonyl, aminocarbonyl, alkylaminocarbonyl, dialkylaminocarbonyl, alkenylaminocarbonyl, dialkenylaminocarbonyl, cycloalkylaminocarbonyl, alkylsulfonylamino, amino sulfonyl, alkylaminosulfonyl, dialkylaminosulfonyl, alkylsulfoximino, aminothiocarbonyl, alkylaminothiocarbonyl or dialkylaminothiocarbonyl, where all the aforementioned radicals may optionally be substituted; or
   represent phenylalkyl, phenoxy, phenylalkyloxy, phenoxyalkyl, phenylthio, phenylthioalkyl, phenylsulfinyl, phenylsulfonyl, hetarylalkyl, hetaryloxy, hetarylalkyloxy, hetarylthio, hetarylsulfinyl or hetarylsulfonyl, where all the aforementioned radicals may optionally be substituted; or
   represent cycloalkylalkyl, cycloalkyloxy, cycloalkylalkoxy, cycloalkylthio, cycloalkylalkylthio, cycloalkylsulfinyl, cycloalkylalkylsulfinyl, cycloalkylsulfonyl, cycloalkylalkylsulfonyl or cycloalkenyl, where all the aforementioned radicals may each optionally be substituted; or
   represent NR′R″,
      where R′ and R″ each independently of one another
      represent hydrogen, cyano, alkyl, haloalkyl, cyanoalkyl, hydroxyalkyl, alkoxyalkyl, alkylthioalkyl, alkenyl, haloalkenyl, cyanoalkenyl, alkynyl, haloalkynyl, cyanoalkynyl, acyl or alkoxycarbonyl; or
      R′ and R″ together with the nitrogen atom to which they are attached may form an optionally substituted, saturated or unsaturated five- to eight-membered ring optionally interrupted by one or more heteroatoms which are selected
      independently from the group consisting of O, S and N; or
   are a 3- to 6-membered saturated, partly saturated or aromatic ring which may optionally contain one to three heteroatoms which are selected independently from the group consisting of O, S and N, and which may optionally be substituted;

or X and z, or Y and z, together with the carbon atoms to which they are attached, form a 5- or 6-membered ring which is optionally substituted and optionally interrupted by one or more heteroatoms which are selected independently from the group consisting of O, S, N and CO.

It has been additionally found that the novel compounds of the formula (I) have good efficacy as pesticides, for example against arthropods and especially insects and acarids, and additionally generally have very good compatibility with plants, especially crop plants, and/or have favourable toxicological and/or environmentally relevant properties.

Preferred substituents or ranges for the radicals shown in the compounds of the formula (I) are illustrated below.
V represents oxygen;
Q represents C—R or nitrogen;
R represents hydrogen, hydroxy, halogen, cyano, alkyl, cycloalkyl, haloalkyl, alkoxy, haloalkoxy, alkylthio, haloalkylthio, alkylsulfinyl, haloalkylsulfinyl, alkylsulfonyl, haloalkylsulfonyl, amino, monoalkylamino or dialkylamino;
Q¹ represents C—R¹ or nitrogen;
Q² represents C—R² or nitrogen;
Q³ represents C—R³ or nitrogen;
Q⁴ represents C—R⁴ or nitrogen;
   where at least one of the structural elements Q¹, Q², Q³, Q⁴ is nitrogen and at most two of the structural elements Q¹, Q², Q³, Q⁴ are nitrogen;
R¹, R², R³, R⁴ each independently of one another represent hydrogen, hydroxy, halogen, cyano, alkyl, cycloalkyl, haloalkyl, alkoxy, haloalkoxy, hetaryl, alkylthio, haloalkylthio, alkylsulfinyl, haloalkylsulfinyl, alkylsulfonyl, haloalkylsulfonyl, amino, monoalkylamino or dialkylamino;
W represents hydrogen or halogen;
n represents the number 0 or 1;
X, Y and z independently of one another
   represent hydrogen, fluorine, chlorine, bromine, cyano, nitro, amino, hydroxyl, alkyl, haloalkyl, alkenyl, alkynyl, alkoxy, haloalkoxy or aminothiocarbonyl;
   or represent benzyl, phenoxy, phenylthio, cyclopropylmethyl, cyclopropyloxy or cyclopropylthio, which is optionally mono- or polysubstituted identically or differently by fluorine, chlorine, bromine, cyano, nitro, hydroxyl, amino, methyl, ethyl, trifluoromethyl, difluoromethyl, trifluoroethyl, difluoroethyl, methoxy, ethoxy, trifluoromethoxy, trifluoroethoxy, or optionally methyl-, fluorine-, chlorine- or cyano-substituted cyclopropyl;
   or represent cyclopropyl, cyclobutyl, cyclopentyl, cyclohexyl or phenyl, which is optionally mono- or polysubstituted identically or differently by fluorine, chlorine, bromine, cyano, nitro, hydroxyl, amino, methyl, ethyl, trifluoromethyl, difluoromethyl, trifluoroethyl, difluoroethyl, methoxy, ethoxy, trifluoromethoxy, trifluoroethoxy, or optionally methyl-, fluorine-, chlorine- or cyano-substituted cyclopropyl.

Particularly preferred substituents or ranges for the radicals shown in the compounds of formula (I) are elucidated below.
V represents oxygen;
Q represents C—R or nitrogen;
R represents hydrogen, halogen, (C₁-C₄)-alkyl, (C₁-C₄)-haloalkyl or (C₃-C₆)-cycloalkyl;
Q¹ represents C—R¹ or nitrogen;
Q² represents C—R² or nitrogen;
Q³ represents C—R³ or nitrogen;
Q⁴ represents C-R⁴ or nitrogen;
   where at least one of the structural elements Q¹, Q², Q³, Q⁴ is nitrogen and at most two of the structural elements Q¹, Q², Q³, Q⁴ are nitrogen;
R¹, R², R³, R⁴ each independently of one another represent hydrogen, halogen, (C₁-C₄)-alkyl, (C₁-C₄)-haloalkyl or (C₃-C₆)-cycloalkyl;
W represents hydrogen or fluorine;
n represents the number 0 or 1;
X and Y each independently represent hydrogen, fluorine, chlorine, bromine, methyl, ethyl, trifluoromethyl, (2,2)-difluoromethyl, difluoromethoxy, trifluoromethoxy, cyclopropyl, cyano, amino, hydroxyl or nitro;
z represents hydrogen.

Likewise very particularly preferred substituents or ranges of the radicals shown in the compounds of the formula (I) are elucidated below.
V represents oxygen;
Q represents C—R or nitrogen;
R represents hydrogen, halogen, (C₁-C₄)-alkyl, (C₁-C₄)-haloalkyl, (C₁-C₄)-alkoxy, (C₁-C₄)-haloalkoxy or (C₃-C₆)-cycloalkyl;
Q¹ represents C—R¹ or nitrogen;
Q² represents C—R² or nitrogen;
Q³ represents C—R³ or nitrogen;
Q⁴ represents C—R⁴ or nitrogen;
   where at least one of the structural elements Q¹, Q², Q³, Q⁴ is nitrogen and at most two of the structural elements Q¹, Q², Q³, Q⁴ are nitrogen;
R¹, R², R³, R⁴ each independently of one another represent hydrogen, halogen, (C₁-C₄)-alkyl, (C₁-C₄)-haloalkyl, (C₁-C₄)-alkoxy, (C₁-C₄)-haloalkoxy or (C₃-C₆)-cycloalkyl;
W represents hydrogen or fluorine;
n represents the number 0 or 1;

X and Y each independently represent hydrogen, fluorine, chlorine, bromine, methyl, ethyl, trifluoromethyl, (2,2)-difluoromethyl, methoxy, difluoromethoxy, trifluoromethoxy, cyclopropyl, cyano, amino, hydroxyl or nitro;
z represents hydrogen.

Very particularly preferred substituents or ranges of the radicals shown in the compounds of the formula (I) are elucidated below.
V represents oxygen;
Q represents C—R or nitrogen;
R represents hydrogen, fluorine, chlorine, methyl, ethyl, tert-butyl, trifluoromethyl, methoxy, trifluoromethoxy or cyclopropyl;
Q¹ represents C—R¹ or nitrogen;
Q² represents C—R² or nitrogen;
Q³ represents C—R³ or nitrogen;
Q⁴ represents C—R⁴ or nitrogen;
   where at least one of the structural elements Q¹, Q², Q³, Q⁴ is nitrogen and at most two of the structural elements Q¹, Q², Q³, Q⁴ are nitrogen;
R¹, R², R³, R⁴ each independently of one another represent hydrogen, fluorine, chlorine, methyl, ethyl, tert-butyl, trifluoromethyl, methoxy, trifluoromethoxy or cyclopropyl;
W represents hydrogen or fluorine;
n represents the number 0 or 1;
X represents hydrogen, chlorine, fluorine or methyl;
Y represents chlorine, bromine, cyano, methyl, trifluoromethyl, fluorine or methoxy;
z represents hydrogen.

It is further preferred among these very particularly preferred substituents or ranges that X and Y represent the following combinations (Y,X): (Me, F), (Me,H), (Me,Cl), (Me,Me), (Cl,Cl), (Cl,F), (CN,F), (MeO,F), (MeO,H), (Cl,H), (Br,H), (Br,F), (CN,H), (F,F), (CF₃,H).

From among these very particularly preferred substituents or ranges of the radicals shown in the compounds of the formula (I), the following are elucidated below.
(1)
V represents oxygen;
Q represents C—R;
R represents hydrogen or methyl;
Q¹ represents nitrogen;
Q² represents C—R²;
Q³ represents C—R³;
Q⁴ represents C—R⁴;
R², R³, R⁴ each represent hydrogen;
W represents fluorine;
n represents the number 0 or 1;
X represents chlorine, fluorine or methyl;
Y represents chlorine or methyl,
   where X and Y especially represent the following (Y,X) combinations: (Me,F), (Me,Me), (Cl,Cl);
z represents hydrogen.
(2)
V represents oxygen;
Q represents C—R;
R represents hydrogen;
Q¹ represents C—R¹;
Q² represents nitrogen;
Q³ represents C—R³;
Q⁴ represents C—R⁴;
R¹, R³, R⁴ each represent hydrogen;
W represents fluorine;
n represents the number 0 or 1;
X represents fluorine;
Y represents methyl,
z represents hydrogen.
(3)
V represents oxygen;
Q represents C—R;
R represents hydrogen;
Q¹ represents C—R¹;
Q² represents C—R²;
Q³ represents nitrogen;
Q⁴ represents C—R⁴;
R¹, R², R⁴ each represent hydrogen;
W represents fluorine;
n represents the number 0 or 1;
X represents fluorine;
Y represents methyl,
z represents hydrogen.
(4)
V represents oxygen;
Q represents C—R;
R represents hydrogen;
Q¹ represents C—R¹;
Q² represents C—R²;
Q³ represents C—R³;
Q⁴ represents nitrogen;
R¹, R², R³ each represent hydrogen;
W represents fluorine;
n represents the number 0 or 1;
X represents fluorine;
Y represents methyl,
z represents hydrogen.

When sulfur and/or nitrogen occur in rings in the above definitions, for example in expressions such as “in which the rings may contain at least one heteroatom from the group of sulfur, oxygen (where oxygen atoms must not be directly adjacent) and nitrogen” or “in which one or two ring members may each be replaced by a heteroatom from the group of sulfur, oxygen (where oxygen atoms must not be directly adjacent) and nitrogen”, unless stated otherwise, the sulfur may also be present in the form of SO or SO₂; the nitrogen, if it is not in the form of —N═, as well as NH, may also be present in the form of N-alkyl (especially N—C₁-C₆-alkyl).

In the broadest and the preferred definitions, unless stated otherwise,

halogen is selected from the group consisting of fluorine, chlorine, bromine and iodine, preferably in turn from the group consisting of fluorine, chlorine and bromine,

hetaryl (synonymous with heteroaryl, including as part of a larger unit, for example hetarylalkyl) is selected from the group of furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl.

In the particularly preferred definitions, unless stated otherwise,

halogen is selected from the group consisting of fluorine, chlorine, bromine and iodine, preferably in turn from the group consisting of fluorine, chlorine and bromine,

hetaryl (also as part of a relatively large unit such as, for example, hetarylalkyl) is selected from the group consisting of furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl.

Halogen-substituted radicals, for example haloalkyl, are mono- or polyhalogenated up to the maximum possible number of substituents. In the case of polyhalogenation, the halogen atoms may be the same or different. Halogen represents fluorine, chlorine, bromine and iodine, especially fluorine, chlorine and bromine.

Saturated or unsaturated hydrocarbon radicals, such as alkyl or alkenyl, may each be straight-chain or branched if possible, including in combination with heteroatoms, as, for example, in alkoxy.

Optionally substituted radicals may be mono- or polysubstituted, where the substituents in the case of polysubstitution may be the same or different.

The radical definitions or elucidations given above in general terms or within preferred ranges apply correspondingly to the end products (including the compounds of the formulae (I-A) to (I-D) shown later), and to the starting materials and intermediates. These radical definitions can be combined with one another as desired, i.e. including combinations between the respective ranges of preference.

Preference according to the invention is given to compounds of the formula (I) which contain a combination of the meanings listed above as being preferred.

Particular preference according to the invention is given to compounds of the formula (I) which contain a combination of the meanings listed above as being particularly preferred.

Very particular preference according to the invention is given to compounds of the formula (I) which contain a combination of the meanings listed above as being very particularly preferred.

In a preferred embodiment, the invention relates to compounds of the formula (I-A)

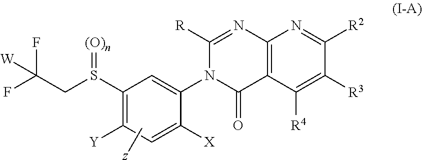

In a further preferred embodiment, the invention relates to compounds of the formula (I-B)

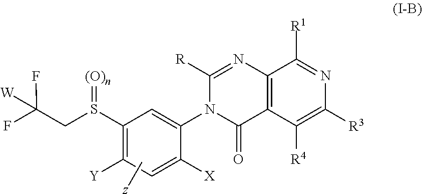

In a further preferred embodiment, the invention relates to compounds of the formula (I-C)

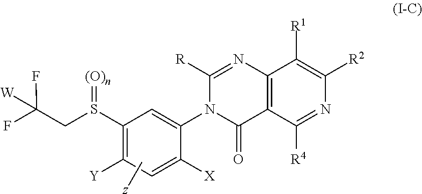

In a further preferred embodiment, the invention relates to compounds of the formula (I-D)

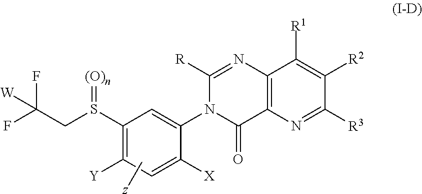

In the formulae (I-A) to (I-D), the radicals or structural elements R, R¹, R², R³, R⁴, W, n, X, Y and z each have the meanings mentioned above.

More particularly, X and Y represent the following (Y,X) combinations: (Me, F), (Me,H), (Me,Cl), (Me,Me), (Cl,Cl) and (Cl,F).

In a further preferred aspect of embodiment (I-A), the invention relates to compounds of the formula (I-A-1)

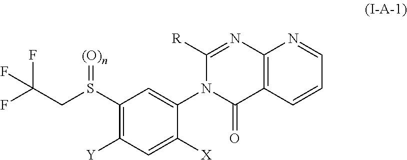

In a further preferred aspect of embodiment (I-B), the invention relates to compounds of the formula (I-B-1)

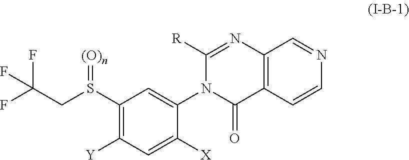

In a further preferred aspect of embodiment (I-C), the invention relates to compounds of the formula (I-C-1)

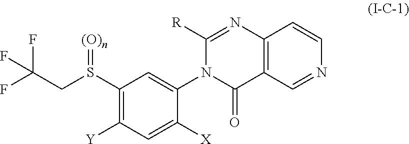

In a further preferred aspect of embodiment (I-D), the invention relates to compounds of the formula (I-D-1)

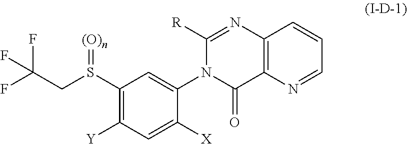

In the formulae (I-A-1) to (I-D-1), the radicals or structural elements R, n, X and Y have the meanings mentioned above.

More particularly, X and Y represent the following (Y,X) combinations: (Me, F), (Me,H), (Me,Cl), (Me,Me), (Cl,Cl) and (Cl,F).

The compounds of the formula (I) according to the invention and the acid addition salts and metal salt complexes thereof have good efficacy, especially for controlling animal pests, which include arthropods and in particular insects and acarids.

The compounds of the formula (I) may possibly also, depending on the nature of the substituents, be in the form of stereoisomers, i.e. in the form of geometric and/or optical isomers or isomer mixtures of varying composition. The present invention provides both the pure stereoisomers and any mixtures of these isomers, even if generally only compounds of the formula (I) are referred to. The compounds of the formula (I) according to the invention can be present in various polymorphic forms or as a mixture of different polymorphic forms. Both the pure polymorphs and the polymorph mixtures form part of the subject-matter of the invention and can be used in accordance with the invention.

However, preference is given in accordance with the invention to using the optically active, stereoisomeric forms of the compounds of the formula (I) and salts thereof.

The invention therefore relates both to the pure enantiomers and diastereomers and to mixtures thereof for controlling animal pests, including arthropods and especially insects and acarids.

Suitable salts of the compounds of the general formula (I) include customary nontoxic salts, i.e. salts with appropriate bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and with inorganic amines, for example triethylammonium, dicyclohexylammonium, N,N′-dibenzylethylenediammonium, pyridinium, picolinium or ethanolammonium salts, salts with inorganic acids, for example hydrochlorides, hydrobromides, dihydrosulfates, trihydrosulfates, or phosphates, salts with organic carboxylic acids or organic sulfonic acids, for example formates, acetates, trifluoroacetates, maleates, tartrates, methanesulfonates, benzenesulfonates or para-toluenesulfonates, salts with basic amino acids, for example arginates, aspartates or glutamates, and the like.

The compounds according to the invention are defined in general terms by the formula (I), which includes all the possible rotamers, tautomers and mixtures thereof. Mention may be made, for example, of the tautomers of the general formula (I) in which Q represents C—R and R represents hydroxy, thiol, amino or alkylamino.

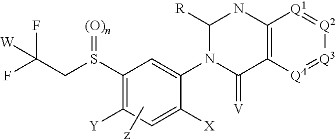

Here, the structural elements V, Q¹, Q², Q³,Q⁴, W, n, X, Y and z have the meanings mentioned above.

The compounds of the formula (I) according to the invention can be prepared by customary methods known to those skilled in the art. Varoius preparation methods, which also form part of the subject matter of the invention, are described in the reaction schemes Process A to Process F below.

Preparation Processes

The compounds of the general formula (I) can be classified into compounds where n=0 (Ia), n=1 (Ib) and n=2 (Ic) and can be prepared according to the scheme below, for example according to Methods A and B, as described in the application WO 2010/100189. In deviation from these methods, the compounds of the formula (I) can also be prepared according to Processes C, D and E.

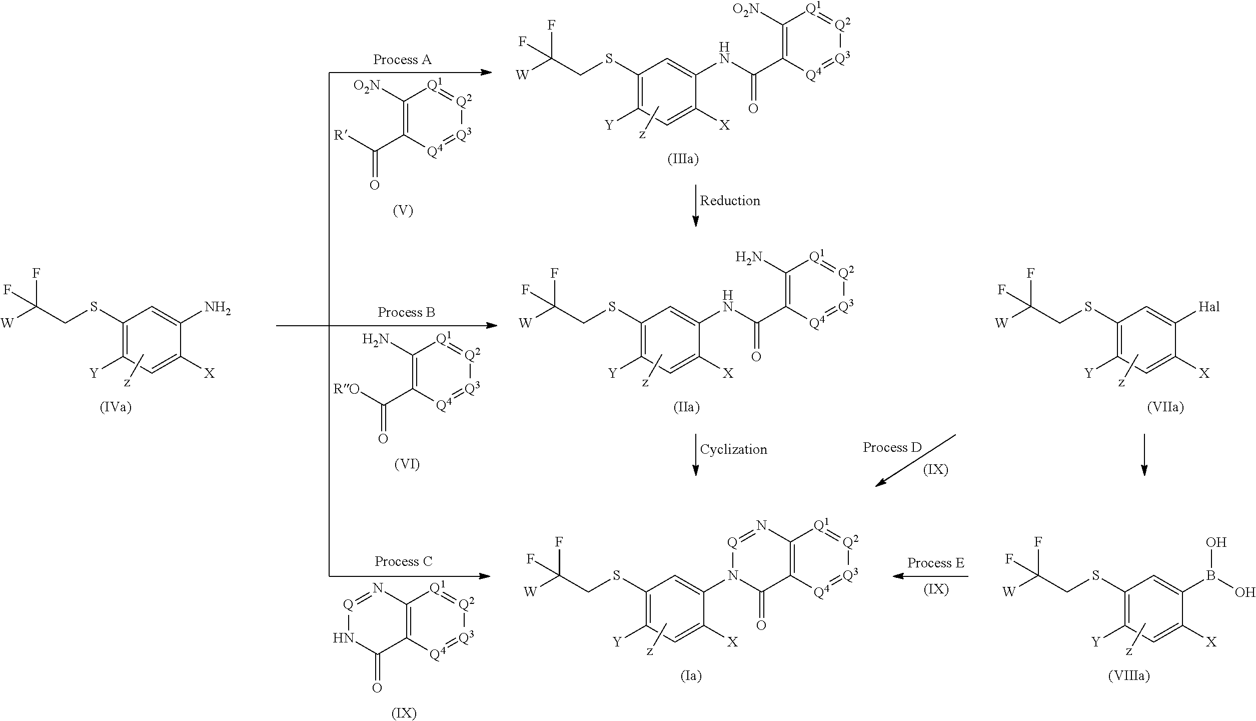

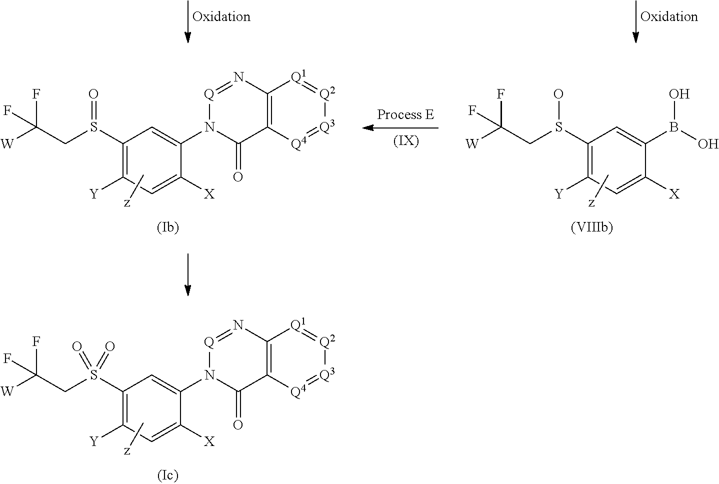

Process A

Compounds of the formula (IIIa) or tautomers thereof can be prepared, for example, according to Process A by reacting anilines of the formula (IVa) with nitro compounds of the formula (V) in which R* represents hydroxy or halogen (preferably Cl and Br).

Many different methods for preparing carboxamides from carboxylic acids (R*=hydroxy) or carbonyl halides (R=halogen) are known, for example G. Benz in Comprehensive Organic Synthesis, 1^{st} Ed., Pergamon Press, Oxford, 1991, Vol. 6, pp. 381-417; P. D. Bailey et al. in Comprehensive Organic Functional Group Transformation, 1st Ed., Elsevier Science Ltd., Oxford, 1995, Vol. 5, pp. 257-308 and R. C. Larock in Comprehensive Organic Transformations, 2nd Ed., Wiley-VCII, New York, Weinheim, 1999, pp. 1929-1994. Carbonyl chlorides can be isolated or used as generated in situ.

The amidation reactions are optionally carried out in the presence of a condensing agent, optionally in the presence of an acid activator, optionally in the presence of an acid acceptor and optionally in the presence of a solvent. Useful condensing agents are all the condensing agents typically usable for such amidation reactions. Examples which may be mentioned are acid halide formers such as phosgene, phosphorus trichloride, oxalyl chloride or thionyl chloride; carbodiimides such as N,N′-dicyclohexylcarbodiimide (DCC) and 1-(3-dimethylaminopropyl)-3-ethylcarbodiimide (EDCI), or other customary condensing agents such as phosphorus pentoxide, polyphosphoric acid, N,N′-carbonyldiimidazole, 2-chloropyridine 1-methoiodide (Mukaiyama's reagent), 2-ethoxy-N-ethoxycarbonyl-1,2-dihydroquinoline (EEDQ), triphenylphosphine/carbon tetrachloride, bromotripyrrolidinophosphonium hexafluorophosphate (BROP), O-(1H-benzotriazol-1-yloxy)tris(dimethylamino)phosphonium hexafluorophosphate (BOP), N,N,N′,N′-bis(tetramethylene)chlorouronium tetrafluoroborate, O-(1H-benzotriazol-1-yl)-N,N,N′,N′-tetramethyluronium hexafluorophosphate (HBTU), O-(1H-benzotriazol-1-yl)-N,N,N′,N′-bis(tetramethylene)uronium hexafluorophosphate, O-(1H-benzotriazol-1-yl)-N,N,N′,N′-tetramethyluronium tetrafluoroborate (TBTU), O-(1H-benzotriazol-1-yl)-N,N,N′,N′-bis(tetramethylene)uronium tetrafluoroborate, O-(7-azabenzotriazol-1-yl)-N,N,N′,N′-tetramethyluronium hexafluorophosphate (HATU) and 1-hydroxybenzotriazole. These reagents can be used separately or, if appropriate, in combination. Useful acid acceptors are all customary inorganic or organic bases, for example triethylamine, diisopropylethylamine, N-methylmorpholine or N,N-dimethylaminopyridine. Process A according to the invention is optionally carried out in the presence of a suitable reaction auxiliary, for example N,N-dimethylformamide or N,N-dimethylaminopyridine.

Furthermore, it is also possible to use mixed anhydrides for preparing (III), as published, for example, in J. Am. Chem. Soc 1967, 5012. In this process, it is possible to use various chloroformic esters, for example isobutyl chloroformate, isopropyl chloroformate. It is likewise possible to use diethylacetyl chloride, trimethylacetyl chloride and the like for this purpose.

Compounds of the general formula (IIa) or tautomers thereof can be prepared, for example, by reduction of the nitro compounds of the general formula (IIIa) according to methods known from the literature. Suitable processes for such reductions are in particular metal-mediated reactions such as, for example, tin(II) chloride, iron powder, zinc powder, Raney nickel, palladium(0) on carbon or platinum dioxide (as hydrate). The metal-mediated reductions, for example with tin(II) chloride, can be carried out according to a process described in Organic Syntheses Coll. Vol. (III), 453.

Process B

Alternatively, compounds of the general formula (IIa) can be prepared by an acylation reaction according to Process B where an aniline of the general formula (IVa) is reacted with a suitable carboxylic acid derivative of the formula (VI), where R** preferably represents alkyl. This can take place without activation, as described by B. M. Trost and I. Fleming in Comprehensive Organic Synthesis, Ed. Pergamon, 1991, Vol. 6. Alternatively, the literature discloses activation methods by formation of an aluminum amide, as by T. Ooi and K. Marouka in Science of Synthesis, Ed. Georg Thieme, 2003, Vol. 7, 225-246. These aluminum amides can be obtained from the anilines or their salts by reaction with trimethylaluminum or their air-stable adduct with 1,4-diazobicyclo[2.2.3]octane (DABCO), as described by S. Woodward in Tet. Lett. 2006, 47, 5767-5769. R** may also represent hydrogen, so that all synthesis methods described in Process A would be suitable for the synthesis of compounds of the formula (IIa).

Various methods are suitable for the preparation of thioethers of the general formula (Ia). Examples which may be mentioned are: starting with compounds of the formula (IIa) by ring closure; starting with anilines of the formula (IVa) by reaction with bicyclic compounds of the formula (IX) according to Process C or starting with halides of the formula (VIIa) or boronic acids of the formula (VIIIa) or (VIIIb) by metal-catalysed reactions according to Process D or Process E.

Cyclization

For Q=C—R, where R represents H or alkyl, the preparation of the thioether of the general formula (Ia) can take place according to methods known from the literature by cyclization of open-chain precursors of the formula (IIa) with an orthoester such as triethyl orthoformate or triethyl orthoacetate, optionally in the presence of a solvent and diluent, optionally in the presence of an acid of organic nature (such as para-toluenesulfonic acid) or inorganic nature (such as hydrochloric acid or sulfuric acid) in catalytic or stoichiometric amounts or in excess or instead of the solvent or diluent.

For Q=C—R, where R represents alkyl or haloalkyl, the preparation of the thioether of the general formula (Ia) can also be by reaction with the appropriate carboxylic anhydrides according to methods known from the literature, for example as described for R═CF3 in the patent WO 2008/039489.

For Q=N, the compounds of the formula (Ia) can be prepared by diazotization of the compounds of the formula (IIa) according to methods known from the literature. For example, a nitrite source such as sodium nitrite or isobutyl nitrite is added to compounds of the formula (IIa), typically in water, alcohol or a polar inert solvent, at from 0 to 5° C. in the presence of an organic or inorganic acid. Exemplary reaction conditions can be found, for example, in the international laid-open publication WO 2004/242572 or in J. Amer. Chem. Soc. Perkin Trans. 1, 1980, 633-638.

Process C

Alternatively, the compounds of the formula (Ia) can be prepared from anilines of the formula (IVa) by reaction with bicyclic compounds of the formula (IX) or their tautomeric hydroxypyrimidinones according to Process C. As described by Yang et al. in Org. Lett. 2009, 11, 6, 1421-1424, the N-arylation of the hydroxypyrimidinones is carried out under mild reaction conditions, for example by HATU-mediated coupling with primary amines using DBU as base in acetonitrile as solvent, in most cases at room temperature or at up to 70° C.

Process D

An alternative preparation of the compounds of the formula (Ia) is provided by the reaction of halides of the formula (VIIa) with bicyclic compounds of the formula (IX) under metal-catalysed reaction conditions. The literature discloses numerous methods, for example in Chem. Pharm. Bull. 1997, 45, 4, 719-721; in Tet. Lett. 2006, 47, 7677-7680; or Synlett 2008, 9, 1335-1340, where the metal source used is copper iodide in the presence of a base and optionally a ligand at elevated temperatures (for example from 120 to 150° C.).

Process E

It has likewise been found that the reaction of boronic acids of the formula (VIIIa) with bicyclic compounds of the formula (IX) by metal-catalysed reactions may serve to prepare the compounds of the formula (Ia). An overview of such reactions can be found in Synthesis 2011, 6, 829-856. A suitable metal source is copper(II) acetate, as in Synlett 2010, 5,721-724; Tetrahedron 2006, 62, 8, 1764-1771; Tetrahedron Lett. 2005, 46, 34, 5699-5702 or WO 2010/104818.

The oxidation of the boronic acids of the formula (VIIIa) or their boronic esters according to methods known from the literature, for example with sodium periodate, leads to sulfoxides of the formula (VIIIb) which can likewise be reacted with bicyclic compounds of the formula (IX) under metal-catalysed reaction conditions, resulting in the target compounds (Ib).

When carrying out the Processes D and E according to the invention, any commercial microwave apparatus suitable for these reactions may optionally be employed (for example Anton Paar Monowave 300, CEM Discover S, Biotage Initiator 60).

Compounds of the general formula (Ib) and (Ic) can be prepared through oxidation by processes known from the literature from compounds of the general formula (Ia), for example by means of an oxidizing agent in a suitable solvent and diluent. Suitable oxidizing agents are, for example, dilute nitric acid, hydrogen peroxide and peroxycarboxylic acids, for example meta-chloroperbenzoic acid. Suitable solvents are inert organic solvents, typically acetonitrile and halogenated solvents such as dichloromethane, chloroform or dichloroethane.

A large number of different methods are suitable for generating enantiomerically enriched sulfoxides, as described by A. R. Maguire in ARKIVOC, 2011(i), 1-110: metal-catalysed asymmetric oxidations of thioethers, for example with titanium and vanadium as the most frequently employed catalyst sources, in the form of Ti(OⁱPr₄) and VO(acac)₂, together with a chiral ligand and an oxidizing agent such as tert-butyl hydroperoxide (TBHP), 2-phenylpropan-2-yl hydroperoxide (CHP) or hydrogen peroxide; non-metal-catalysed asymmetric oxidations employing chiral oxidizing agents or chiral catalysts; electrochemical or biological asymmetric oxidations and also kinetic resolution of sulfoxides and nucleophilic shift (according to Andersen's method).

The enantiomers can also be obtained from the racemate, for example by separating them on a preparative scale by chiral HPLC.

Alternatively, compounds of the general formula (Ib) may be prepared by methods similar to those mentioned here carried out in a different order, for example by oxidation of the anilines of the formula (IVa) to give sulfoxides of the formula (IVb) and their further conversion according to Process A, B or C.

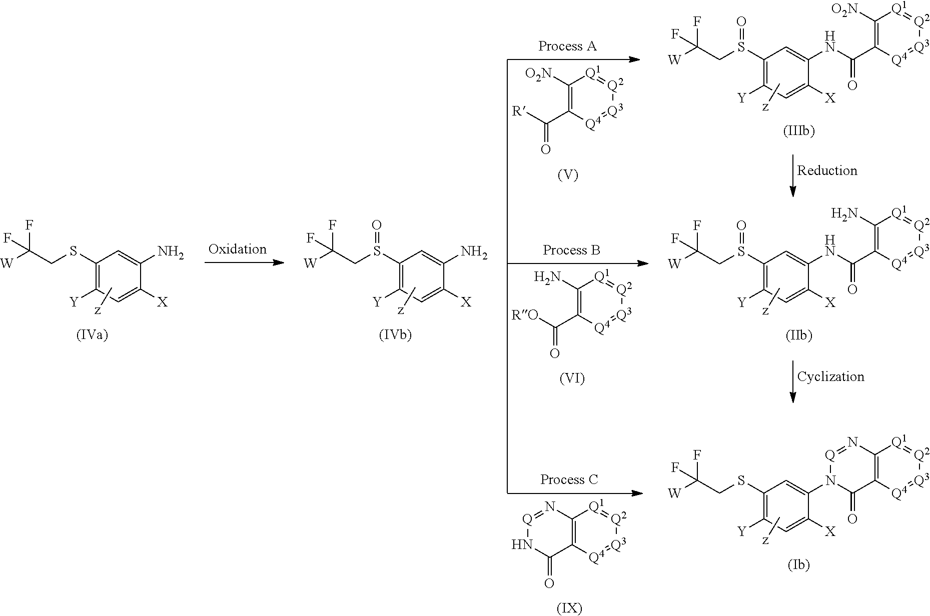

In deviation from these methods, the compounds of the formula (I) where Q=C—R can also be prepared according to Process F.

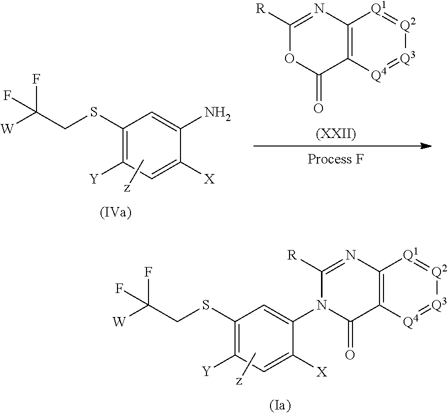

Anilines of the formula (IVa) can be reacted with oxazinones of the formula to give target compounds of the formula (Ia), for example according to Assiut Univ. J. of Chemistry 2006, 45-63. Oxazinones of the formula (XXII) are known or can be prepared by methods known from the literature, for example as in Journal of the Chemical Society 1965, 4240-4246.

Reactions in the Microwave

In the performance of the processes according to the invention, it is optionally possible to use any commercial microwave apparatus suitable for these reactions (e.g. Anton Paar Monowave 300, CEM Discover S, Biotage Initiator 60).

Description of the Starting Materials and Intermediates

Anilines of the formula (IV), halides of the formula (VII) and boronic acids of the formula (VIII) are central building blocks for preparing the compounds of the formula (I).

The anilines of the general formula (IV) can be classified into compounds where n=0 (IVa) and n=1 (IVb).

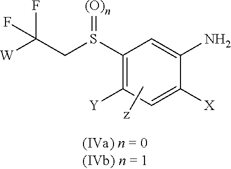

Some of the anilines of the formula (IVa) are known from the literature, for example from JP 2007/284356, or they can be synthesized by processes known from the literature.

The anilines of the general formula (IVa) can be prepared, for example, as in the scheme below;

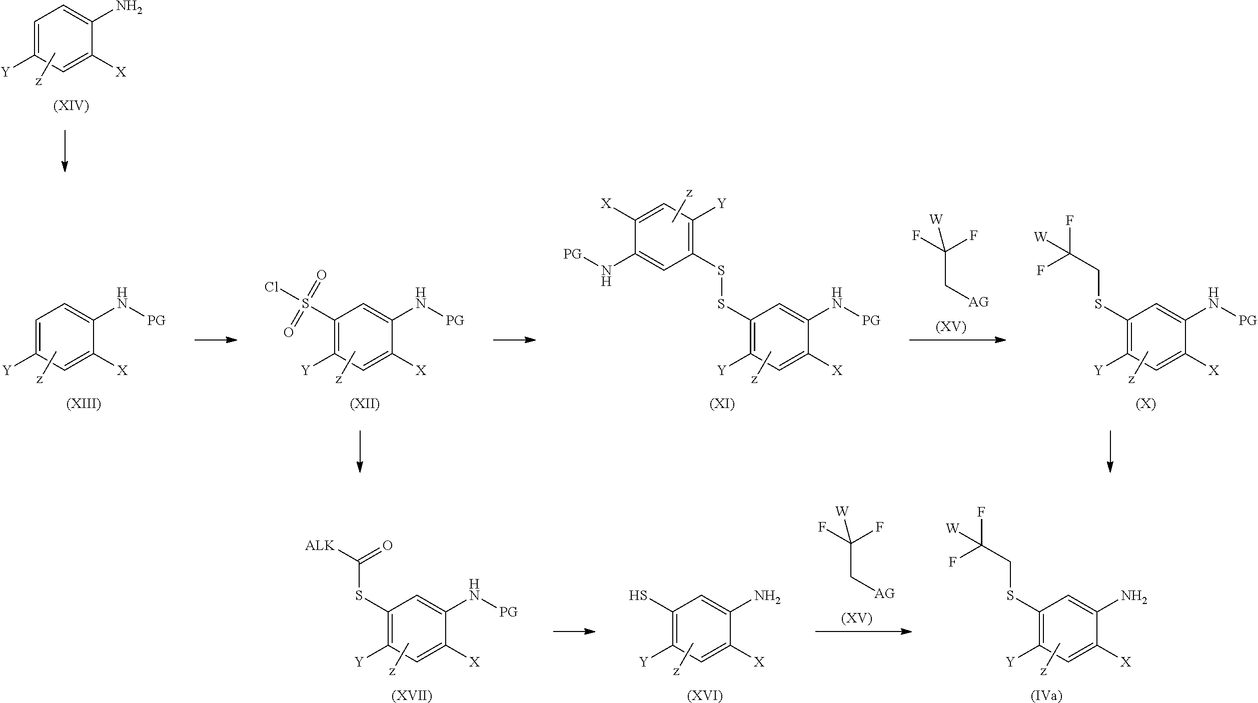

where X, Y, Z and W are as defined above, AG is a leaving group and PG is a protecting group.

Anilines of the formula (XIV) are either commercially available or can be prepared by known methods. They can be protected with a suitable protective group, for example an acetyl group, to give compounds of the formula (XIII). In the presence of acids, acid anhydrides or acid chlorides, for example, the anilines (XIV) can be converted to the corresponding anilides (XIII). The chlorosulfonation of the protected anilines (XIII) with chlorosulfonic acid gives the corresponding sulfonyl chlorides (XII). The reduction of the sulfonyl chlorides (XII) to the disulfides (XI) is possible by methods known from the literature, for example iron in hydrochloric acid or iodide. The reaction of the disulfides (XI) with haloalkyl electrophiles of the formula (XV) where AG is a leaving group, for example chlorine, bromine, tosylate, mesylate or triflate, gives the sulfides (X). The protecting group can be removed by suitable methods known from the literature, so as to obtain anilines of the formula (IVa).

Instead of the reduction to the disulfide (XI), the sulfonyl chloride (XII) can be reduced with a suitable reducing agent, for example iodine/phosphorus, to give the alkyl thioate (XVII), and then deprotected by a suitable method, for example the reaction with potassium hydroxide solution, to give thiols of the formula (XVI). Reaction of the thiols (XVI) with haloalkyl electrophiles of the formula (XV) where AG represents a leaving group such as, for example, chlorine, bromine, tosylate, mesylate or triflate affords the sulfides (IVa).

Likewise preferably, the thioethers of the formula (IVa) can alternatively be prepared according to the following scheme:

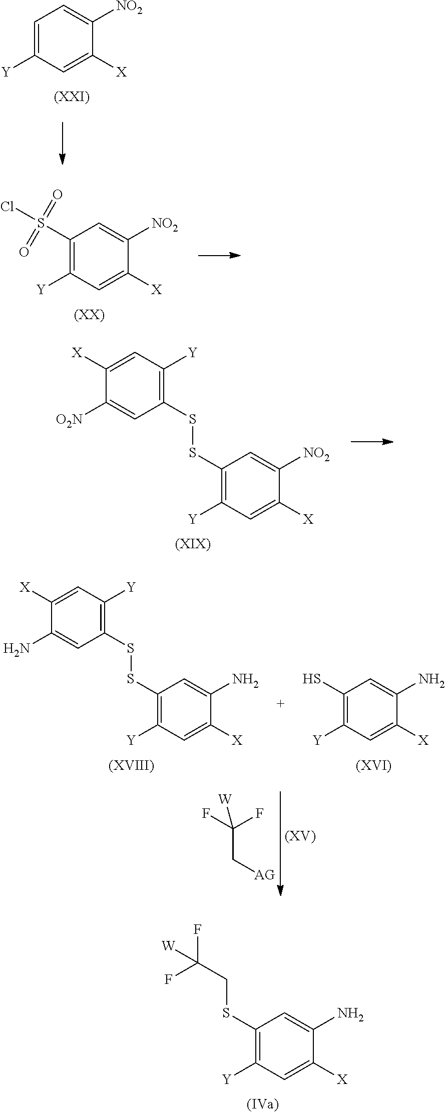

where X, Y, Z and W are as defined above, AG is a leaving group and PG is a protecting group.

The chlorosulfonation of the nitroaromatics of the formula (XXI) with chlorosulfonic acid gives the corresponding sulfonyl chlorides (XX). The reduction of the sulfonyl chlorides (XX) to the bis(nitroaryl) disulfides (XIX) is possible by methods known from the literature, for example iodide. The reduction of the disulfides (XXI) to the disulfanediyldianilines (XIX), some of which are formed as a mixture with the corresponding aminoarylthiols (XVI), is possible with commonly known reducing agents, for example hydrogen, optionally with the aid of heterogeneous catalysts, for example, Raney nickel, platinum on activated carbon or palladium on activated carbon. Reaction of the disulfides (XVIII) or thiophenols (XVI) with haloalkyl electrophiles of the formula (XV) where AG represents a leaving group such as, for example, chlorine, bromine, iodine, tosylate, mesylate or triflate affords the 3(2,2,2-trifluoroethyl)sulfanyl]anilines of the formula (IVa).

Halides of the general formula (VIIa)

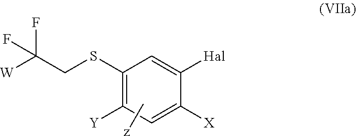

in which X, Y, Z and W have the meanings given above and Hal represents chlorine, bromine or iodine are known from the literature, from WO 2007/034755, JP 2007/081019, JP 2007/284385, JP 2008/260706, JP 2008/308448, JP 2009/023910 or WO 2012/176856, or can be synthesized by processes known from the literature, which may optionally be slightly modified.

Suitable starting materials for the synthesis of the iodides of the general formula (VIIa) are bromides having the same formula, for example in halogen exchange reactions according to methods known from the literature, if appropriate with metal catalysis (see H. Suzuki, Chem. Lett. 1985, 3, 411-412; S. L. Buchwald, J. Amer. Chem. Soc. 2002, 124 (50), 14844-14845). Synthesis is likewise possible proceeding from anilines of the formula (IVa) under Sandmeyer reaction conditions, as described by E. B. Merkushev in Synthesis 1988, 12, 923-937.

Boronic acids of the general formula (VIIIa) and (VIIIb)

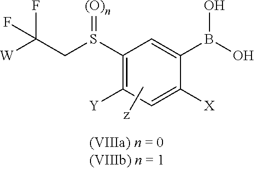

in which X, Y, Z and W have the meanings given above are known from the literature, for example from WO2007/034755, JP2007/284385, JP2009/023910 and WO2012/176856, or can be synthesized by processes known from the literature.

The active compounds according to the invention, or those to be used in accordance with the invention, given good plant tolerance, favourable homeotherm toxicity and good environmental compatibility, are suitable for protecting plants and plant organs, for increasing harvest yields, for improving the quality of the harvested material and for controlling animal pests, especially insects, arachnids, helminths, nematodes and molluscs, which are encountered in agriculture, in horticulture, in animal husbandry, in forests, in gardens and leisure facilities, in the protection of stored products and of materials, and in the hygiene sector. They can preferably be used as crop protection agents. They are effective against normally sensitive and resistant species and against all or some stages of development. The abovementioned pests include:

pests from the phylum of the Arthropoda, more particularly from the class of the Arachnida, for example Acarus spp., Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vaejovis spp., Vasates lycopersici.;

from the class of the Chilopoda, for example Geophilus spp., Scutigera spp.;

from the order or the class of the Collembola, for example Onychiurus armatus.;

from the class of the Diplopoda, for example, Blaniulus guttulatus;

from the class of the Insecta, for example from the order of the Blattodea, for example Blattella asahinai, Blattella germanica, Blatta orientalis, Leucophaea maderae, Panchlora spp., Parcoblatta spp., Periplaneta spp., Supella longipalpa;

from the order of the Coleoptera, for example Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp., Dichocrocis spp., Dicladispa armigera, Diloboderus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Necrobia spp., Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Sitophilus spp., Sitophilus oryzae, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., Zabrus spp.;

from the order of the Diptera, for example Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomyia spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp., Oestrus spp., Oscinella frit, Paratanytarsus spp., Paralauterborniella subcincta, Pegomyia spp., Phlebotomus spp., Phorbia spp., Phormia spp., Piophila casei, Prodiplosis spp., Psila rosae, Rhagoletis spp., Sarcophaga spp., Simulium spp., Stomoxys spp., Tabanus spp., Tetanops spp., Tipula spp.;

from the order of the Heteroptera, for example Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptocorisa varicornis, Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.;

from the order of the Homoptera, e.g. Acizzia acaciaebaileyanae, Acizzia dodonaeae, Acizzia uncatoides, Acrida turrita, Acyrthosipon spp., Acrogonia spp., Aeneolamia spp., Agonoscena spp., Aleyrodes proletella, Aleurolobus barodensis, Aleurothrixus floccosus, Allocaridara malayensis, Amrasca spp., Anuraphis cardui, Aonidiella spp., Aphanostigma pini, Aphis spp., Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes citri, Diaphorina citri, Diaspis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Macrosteles facifrons, Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nettigoniclla spectra, Nilaparvata lugens, Oncometopia spp., Orthezia praelonga, Oxya chinensis, Pachypsylla spp.,

Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.;

from the order of the Hymenoptera, for example Acromyrmex spp., Athalia spp., Atta spp., Diprion spp., Hoplocampa spp., Lasius spp., Monomorium pharaonis, Sirex spp., Solenopsis invicta, Tapinoma spp., Urocerus spp., Vespa spp., Xeris spp.;

from the order of the Isopoda, for example, Armadillidium vulgare, Oniscus asellus, Porcellio scaber;

from the order of the Isoptera, for example, Coptotermes spp., Cornitermes cumulans, Cryptotermes spp., Incisitermes spp., Microtermes obesi, Odontotermes spp., Reticulitermes spp.;

from the order of the Lepidoptera, for example Achroia grisella, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamstra brassicae, Melanitis leda, Mocis spp., Monopis obviella, Mythimna separata, Nemapogon cloacellus, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., Tryporyza incertulas, Tuta absoluta, Virachola spp.;

from the order of the Orthoptera or Saltatoria, for example, Acheta domesticus, Dichroplus spp., Gryllotalpa spp., Hieroglyphus spp., Locusta spp., Melanoplus spp., Schistocerca gregaria; from the order of the Phthiraptera, for example, Damalinia spp., Haematopinus spp., Linognathus spp., Pediculus spp., Phylloera vastatrix, Phtirus pubis, Trichodectes spp.;

from the order of the Psocoptera, for example, Lepinotus spp., Liposcelis spp.;

from the order of the Siphonaptera, for example, Ceratophyllus spp., Ctenocephalides spp., Pulex irritans, Tunga penetrans, Xenopsylla cheopsis;

from the order of the Thysanoptera, for example, Anaphothrips obscurus, Baliothrips biformis, Drepanothrips reuteri, Enneothrips flavens, Frankliniella spp., Heliothrips spp., Hercinothrips femoralis, Rhipiphorothrips cruentatus, Scirtothrips spp., Taeniothrips cardamomi, Thrips spp.;

from the order of the Zygentoma (=Thysanura), for example, Ctenolepisma spp., Lepisma saccharina, Lepismodes inquilinus, Thermobia domestica;

from the class of the Symphyla, for example, Scutigerella spp.;

pests from the phylum of the Mollusca, especially from the class of the Bivalvia, for example Dreissena spp., and from the class of the Gastropoda, for example Anon spp., Biomphalaria spp., Bulinus spp., Deroceras spp., Galba spp., Lymnaea spp., Oncomelania spp., Pomacea spp., Succinea spp.; animal parasites from the phyla of the Plathelminthes and Nematoda, for example Ancylostoma duodenale, Ancylostoma ceylanicum, Ancylostoma braziliensis, Ancylostoma spp., Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Strongyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti;

plant pests from the phylum of the Nematoda, i.e. plant-parasitic nematodes, more particularly Aphelenchoides spp., Bursaphelenchus spp., Ditylenchus spp., Globodera spp., Heterodera spp., Longidorus spp., Meloidogyne spp., Pratylenchus spp., Radopholus spp., Trichodorus spp., Tylenchulus spp., Xiphinema spp., Helicotylenchus spp., Tylenchorhynchus spp., Scutellonema spp., Paratrichodorus spp., Meloinema spp., Paraphelenchus spp., Aglenchus spp., Belonolaimus spp., Nacobbus spp., Rotylenchulus spp., Rotylenchus spp., Neotylenchus spp., Paraphelenchus spp., Dolichodorus spp., Hoplolaimus spp., Punctodera spp., Criconemella spp., Quinisulcius spp., Hemicycliophora spp., Anguina spp., Subanguina spp., Hemicriconemoides spp., Psilenchus spp., Pseudohalenchus spp., Criconemoides spp., Cacopaurus spp.

In addition, it is possible to control, from the sub-kingdom of the Protozoa, the order of the Coccidia, for example Eimeria spp.

The present invention further relates to formulations and use forms prepared therefrom as crop protection compostitons and/or pesticides, for example drench, drip and spray liquors, comprising at least one of the active compounds according to the invention. The use forms optionally comprise further crop protection agents and/or pesticides and/or action-improving adjuvants, such as penetrants, e.g. vegetable oils, for example rapeseed oil, sunflower oil, mineral oils, for example paraffin oils, alkyl esters of vegetable fatty acids, for example rapeseed oil methyl ester or soybean oil methyl ester, or alkanol alkoxylates, and/or spreaders, for example alkylsiloxanes, and/or salts, for example organic or inorganic ammonium or phosphonium salts, for example ammonium sulfate or diammonium hydrogenphosphate, and/or retention promoters, for example dioctyl sulfosuccinate or hydroxypropyl guar polymers, and/or humectants, for example glycerol, and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more active compounds according to the invention, optionally comprise further agrochemical active compounds.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which improves the biological activity of the formulation without having biological activity itself. Examples of adjuvants are agents which promote retention, the spreading characteristics, adhesion to the leaf surface or penetration.

These formulations are produced in a known manner, for example by mixing the active compounds with auxiliaries, for example extenders, solvents and/or solid carriers and/or further auxiliaries, for example surfactants. The formulations are produced either in suitable facilities or else before or during application.

Auxiliaries used may be those substances which are suitable for imparting particular properties, such as particular physical, technical and/or biological properties, to the formulation of the active compound or to the use forms prepared from these formulations (for example ready-to-use crop protection compositions such as spray liquors or seed-dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

In principle, it is possible to use all suitable solvents. Examples of suitable solvents are aromatic hydrocarbons such as xylene, toluene or alkylnaphthalenes, chlorinated aromatic or aliphatic hydrocarbons such as chlorobenzene, chloroethylene or methylene chloride, aliphatic hydrocarbons such as cyclohexane, paraffins, petroleum fractions, mineral and vegetable oils, alcohols such as methanol, ethanol, isopropanol, butanol or glycol and their ethers and esters, ketones such as acetone, methyl ethyl ketone, methyl isobutyl ketone or cyclohexanone, strongly polar solvents such as dimethyl sulfoxide, and water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic minerals such as finely divided silica, alumina and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Especially suitable are those extenders or carriers which are gaseous at standard temperature and under standard pressure, for example aerosol propellants such as halogenated hydrocarbons, and also butane, propane, nitrogen and carbon dioxide.

Examples of emulsifiers and/or foam formers, dispersants or wetting agents with ionic or nonionic properties, or mixtures of these surfactants, are salts of polyacrylic acid, salts of lignosulfonic acid, salts of phenolsulfonic acid or naphthalenesulfonic acid, polycondensates of ethylene oxide with fatty alcohols or with fatty acids or with fatty amines, with substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkyl sulfonates, alkyl sulfates, arylsulfonates, protein hydrolyzates, lignosulfite waste liquors and methylcellulose. The presence of a surfactant is advantageous when one of the active compounds and/or one of the inert carriers is insoluble in water and when application is carried out in water.

Further auxiliaries which may be present in the formulations and the use forms derived therefrom are dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve the chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further possible auxiliaries are mineral and vegetable oils.

Optionally, further auxiliaries may be present in the formulations and the use forms derived therefrom. Examples of such additives include fragrances, protective colloids, binders, adhesives, thickeners, thixotropic agents, penetrants, retention promoters, stabilizers, sequestrants, complexing agents, humectants, spreaders. In general, the active compounds can be combined with any solid or liquid additive which is commonly used for formulation purposes.

Useful retention promoters include all those substances which reduce dynamic surface tension, for example dioctyl sulfosuccinate, or increase viscoelasticity, for example hydroxypropylguar polymers.

Useful penetrants in the present context are all those substances which are typically used to improve the penetration of agrochemically active compounds into plants. Penetrants are defined in this context by their ability to penetrate from the (generally aqueous) application liquor and/or from the spray coating into the cuticle of the plant and hence increase the mobility of the active compounds in the cuticle. The method described in the literature (Baur et al., 1997, Pesticide Science 51, 131-152) can be used for determining this property. Examples include alcohol alkoxylates, for example coconut fat ethoxylate (10) or isotridecyl ethoxylate (12), fatty acid esters, for example rapeseed oil methyl ester or soya oil methyl ester, fatty amine alkoxylates, for example tallowamine ethoxylate (15) or ammonium salts and/or phosphonium salts, for example ammonium sulfate or diammonium hydrogenphosphate.

The formulations contain preferably between 0.00000001% and 98% by weight of active compound or more preferably between 0.01% and 95% by weight of active compound, more preferably between 0.5% and 90% by weight of active compound, based on the weight of the formulation.

The active compound content of the use forms (crop protection compositions) prepared from the formulations can vary within wide limits The active compound concentration of the use forms may typically be between 0.00000001% and 95% by weight of active compound, preferably between 0.00001% and 1% by weight, based on the weight of the use form. Application is accomplished in a customary manner appropriate to the use forms.

The active compounds according to the invention or to be used according to the invention can be used as such or in formulations thereof, including in a mixture with one or more suitable fungicides, bactericides, acaricides, molluscicides, nematicides, insecticides, microbiologicals, useful organisms, fertilizers, bird repellants, phytotonics, sterilants, synergists, safeners, semiochemicals and/or plant growth regulators, in order thus, for example, to broaden the spectrum of action, to prolong the duration of action, to increase the rate of action, to prevent repulsion or prevent evolution of resistance. In addition, active compound combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products. Combination of the active compounds according to the invention, or those for use in accordance with the invention, with mixing partners gives synergistic effects, meaning that the efficacy of the particular mixture is greater than expected on the basis of the efficacies of the individual components. It is generally possible to use the combinations in premixes, tankmixes or readymixes, and also in seed applications.

The active compounds mentioned here under their “common names” are known and are described for example in The Pesticide Manual, 14th Ed., British Crop Protection Council 2006, or can be searched for on the Internet (e.g. http://www.alanwood.net/pesticides).

Insecticides/acaricides/nematicides suitable as mixing partners are: (1) Acetylcholinesterase (AChE) inhibitors, for example

carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or

organophosphates, for example acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl)salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion.

(2) GABA-gated chloride channel antagonists, for example

cyclodiene organochlorines, for example chlordane and endosulfan; or

phenylpyrazoles (fiproles), for example ethiprole and fipronil.

(3) Sodium channel modulators/voltage-dependent sodium channel blockers, for example

pyrethroids, for example acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrine (pyrethrum), resmethrin, silafluofen, tefluthrin, tetramethrin, tetramethrin [(1R) isomers], tralomethrin and transfluthrin; or

DDT; or methoxychlor.

(4) Nicotinergic acetylcholine receptor (nAChR) agonists, for example

neonicotinoids, for example acetamiprid, clothianidin, dinotefuran, imidacloprid, nitenpyram, thiacloprid and thiamethoxam; or

nicotine; or

sulfoxaflor.

(5) Nicotinergic acetylcholine receptor (nAChR) allosteric activators, for example

spinosyns, for example spinetoram and spinosad.

(6) Chloride channel activators, for example

avermectins/milbemycins, for example abamectin, emamectin benzoate, lepimectin and milbemectin.

(7) Juvenile hormone imitators, for example

juvenile hormone analogs, for example hydroprene, kinoprene and methoprene; or

fenoxycarb; or pyriproxyfen.

(8) Active compounds having unknown or nonspecific mechanisms of action, for example

alkyl halides, for example methyl bromide and other alkyl halides; or

chloropicrin; or sulfuryl fluoride; or borax; or tartar emetic.

(9) Selective antifeedants, for example pymetrozine; or flonicamid.

(10) Mite growth inhibitors, for example clofentezine, hexythiazox and diflovidazin; or

etoxazole.

(11) Microbial disruptors of the insect gut membrane, e.g. Bacillus thuringiensis subspecies israelensis, Bacillus thuringiensis subspecies aizawai, Bacillus thuringiensis subspecies kurstaki, Bacillus thuringiensis subspecies tenebrionis and B.t. plant proteins: Cry1Ab, Cry1Ac, Cry1Fa, Cry1A.105, Cry2Ab, Vip3A, mCry3A, Cry3Ab, Cry3Bb, Cry34 Ab1/35Ab1; or

Bacillus sphaericus.

(12) Oxidative phosphorylation inhibitors, ATP disruptors, for example diafenthiuron; or

organotin compounds, e.g. azocyclotin, cyhexatin and fenbutatin oxide; or

propargite; or tetradifon.

(13) Oxidative phosphorylation decouplers that interrupt the H proton gradient, for example chlorfenapyr, DNOC and sulfluramid.

(14) Nicotinergic acetylcholine receptor antagonists, for example bensultap, cartap hydrochloride, thiocyclam, and thiosultap-sodium.

(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Chitin biosynthesis inhibitors, type 1, for example buprofezin.

(17) Molting disruptors, dipteran, for example cyromazine.

(18) Ecdysone receptor agonists, for example chromafenozide, halofenozide, methoxyfenozide and tebufenozide.

(19) Octopaminergic agonists, for example amitraz.

(20) Complex-III electron transport inhibitors, for example hydramethylnon; or acequinocyl; or fluacrypyrim.

(21) Complex-I electron transport inhibitors, for example

METI acaricides, e.g. fenazaquin, fenpyroximate, pyrimidifen, pyridaben, tebufenpyrad and tolfenpyrad; or

rotenone (Derris).

(22) Voltage-gated sodium channel blockers, for example indoxacarb; or metaflumizone.

(23) Inhibitors of acetyl-CoA carboxylase, for example

tetronic and tetramic acid derivatives, e.g. spirodiclofen, spiromesifen and spirotetramat.

(24) Complex-IV electron transport inhibitors, for example

phosphines, for example aluminum phosphide, calcium phosphide, phosphine and zinc phosphide; or

cyanide.

(25) Complex-II electron transport inhibitors, for example cyenopyrafen and cyflumetofen.

(28) Ryanodine receptor effectors, for example

diamides, e.g. chlorantraniliprole, cyantraniliprole and flubendiamide.

Further active compounds, for example amidoflumet, azadirachtin, benclothiaz, benzoximate, bifenazate, bromopropylate, chinomethionat, cryolite, dicofol, diflovidazin, fluensulfone, flufenerim, flufiprole, fluopyram, fufenozide, imidaclothiz, iprodione, meperfluthrin, pyridalyl, pyrifluquinazon, tetramethylfluthrin and iodomethane; and additionally preparations based on Bacillus firmus (particularly strain CNCM I-1582, for example VOTiVO™, BioNem), and the following compounds:

3-bromo-N-{2-bromo-4-chlor-6-[(1-cyclopropylethyl)carbamoyl]phenyl}-1-(3-chloropyridin-2-yl)-1H-pyrazole-5-carboxamide (known from WO 2005/077934), 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), flupyradifurone, 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EP A 0 539 588), 4-{[(6-chloropyrid-3-yl)methyl](methyl)amino}furan-2(5H)-one (known from EP A 0 539 588), {[1-(6-chloropyridin-3-yl)ethyl](methyl)oxido-λ⁴-sulfanylidene}cyanamide (known from WO2007/149134) and its diastereomers {[(1R)-1-(6-chloropyridin-3-yl)ethyl](methyl)oxido-λ⁴-sulfanylidene}cyanamide (A) and {[(1S)-1-(6-chloropyridin-3-yl)ethyl](methyl)oxido-λ⁴-sulfanylidene}cyanamide (B) (likewise known from WO 2007/149134) and also diastereomers [(R)-methyl(oxido){(1R)-1-[6-(trifluoromethyl)pyridin-3-yl]ethyl}-λ⁴-sulfanylidene]cyanamide (A1) and [(S)-methyl(oxido){(1S)-1-[6-(trifluoromethyl)pyridin-3-yl]ethyl}-λ⁴-sulfanylidene]cyanamide (A2), identified as diastereomer group A (known from WO 2010/074747, WO 2010/074751), [(R)-methyl(oxido){(1S)-1-[6-(trifluoromethyl)pyridin-3-yl]ethyl}-λ⁴-sulfanylidene]cyanamide (B1) and [(S)-methyl(oxido){(1R)-1-[6-(trifluoromethyl)pyridin-3-yl]ethyl}-λ⁴-sulfanylidene]cyanamide (B2), identified as diastereomer group B (likewise known from WO 2010/074747, WO 2010/074751) and 11-(4-chloro-2,6-dimethylphenyl)-12-hydroxy-1,4-dioxa-9-azadispiro[4.2.4.2]tetradec-11-en-10-one (known from WO 2006/089633), 3-(4′-fluoro-2,4-dimethylbiphenyl-3-yl)-4-hydroxy-8-oxa-1-azaspiro[4.5]dec-3-en-2-one (known from WO 2008/067911), 1-{2-fluoro-4-methyl-5-[(2,2,2-trifluoroethyl)sulfinyl]phenyl}-3-(trifluoromethyl)-1H-1,2,4-triazol-5-amine (known from WO 2006/043635), afidopyropen (known from WO 2008/066153), 2-cyano-3-(difluoromethoxy)-N,N-dimethylbenzolsulfonamide (known from WO 2006/056433), 2-cyano-3-(difluoromethoxy)-N-methylbenzolsulfonamide (known from WO 2006/100288), 2-cyano-3-(difluoromethoxy)-N-ethylbenzenesulfonamide (known from WO 2005/035486), 4-(difluoromethoxy)-N-ethyl-N-methyl-1,2-benzothiazol-3-amine-1,1-dioxide (known from WO 2007/057407), N-[1-(2,3-dimethylphenyl)-2-(3,5-dimethylphenyl)ethyl]-4,5-dihydro-1,3-thiazol-2-amine (known from WO 2008/104503), {1′-[(2E)-3-(4-chlorophenyl)prop-2-en-1-yl]-5-fluorospiro[indo-3,4′-piperidine]-1(2H)-yl}(2-chloropyridin-4-yl)methanone (known from WO 2003/106457), 3-(2,5-dimethylphenyl)-4-hydroxy-8-methoxy-1, 8-diazaspiro[4.5]dec-3-en-2-one (known from WO 2009/049851), 3-(2,5-dimethylphenyl)-8-methoxy-2-oxo-1, 8-diazaspiro[4.5]dec-3-en-4-yl ethyl carbonate (known from WO 2009/049851), 4-(but-2-yn-1-yloxy)-6-(3,5-dimethylpiperidin-1-yl)-5-fluoropyrimidine (known from WO 2004/099160), (2,2,3,3,4,4,5,5-octafluoropentyl)(3,3,3-trifluoropropyl)malononitrile (known from WO 2005/063094), (2,2,3,3,4,4,5,5-octafluoropentyl)(3,3,4,4,4-pentafluorobutyl)malononitrile (known from WO 2005/063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO 2007/040280), flometoquin, PF1364 (CAS Reg. No. 1204776-60-2) (known from JP 2010/018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO 2007/075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO 2007/075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO 2005/085216), 4-{[(6-chloropyridin-3-yl)methyl](cyclopropyl)amino}-1,3-oxazol-2(5H)-one, 4-{[(6-chloropyridin-3-yl)methyl](2,2-difluoroethyl)amino}-1,3-oxazol-2(5H)-one, 4-{[(6-chloropyridin-3-yl)methyl](ethyl)amino}-1,3-oxazol-2(5H)-one, 4-{[(6-chloropyridin-3-yl)methyl](methyl)amino}-1,3-oxazol-2(5H)-one (all known from WO 2010/005692), pyflubumide (known from WO 2002/096882), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO 2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO 2005/085216), methyl 2-[2-({[3-bromo-1 -(3-chloropyridin-2-yl)-1H-pyrazol-5-yl]carbonyl}amino)-5-cyano-3-methylbenzoyl]-2-methylhydrazinecarboxylate (known from WO 2005/085216), methyl 2-[3,5-dibromo-2-({[3-bromo-1-(3-chloropyridin-2-yl)-1H-pyrazol-5-yl]carbonyl}amino)benzoyl]-1,2-diethylhydrazinecarboxylate (known from WO 2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO 2005/085216), (5RS,7RS;5RS,7SR)-1-(6-chloro-3-pyridylmethyl)-1,2,3,5,6,7-hexahydro-7-methyl-8-nitro-5-propoxyimidazo[1,2-a]pyridine (known from WO 2007/101369), 2-{6-[2-(5-fluoropyridin-3-yl)-1,3-thiazol-5-yl]pyridin-2-yl}pyrimidine (known from WO 2010/006713), 2-{6-[2-(pyridin-3-yl)-1,3-thiazol-5-yl]pyridin-2-yl}pyrimidine (known from WO 2010/006713), 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide (known from WO 2010/069502), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO 2010/069502), N-[2-(tert-butylcarbamoyl)-4-cyano-6-methylphenyl]-1-(3-chloropyridin-2-yl)-3-{[5-(trifluoromethyl)-1H-tetrazol-1-yl]methyl}-1H-pyrazole-5-carboxamide (known from WO 2010/069502), N-[2-(tert-butylcarbamoyl)-4-cyano-6-methylphenyl]-1-(3-chloropyridin-2-yl)-3-{[5-(trifluoromethyl)-2H-tetrazol-2-yl]methyl}-1H-pyrazole-5-carboxamide (known from WO 2010/069502), (1E)-N-[(6-chloropyridin-3-yl)methyl]-N′-cyano-N-(2,2-difluoroethyl)ethanimideamide (known from WO 2008/009360), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN 102057925), methyl 2-[3,5-dibromo-2-({[3-bromo-1-(3-chloropyridin-2-yl)-1H-pyrazol-5-yl]carbonyl}amino)benzoyl]-2-ethyl-1-methylhydrazinecarboxylate (known from WO 2011/049233), heptafluthrin, pyriminostrobin, flufenoxystrobin and 3-chloro-N²-(2-cyanopropan-2-yl)-N¹-[4-(1,1,1,2,3,3,3-heptafluoropropan-2-yl)-2-methylphenyl]phthalamide (known from WO 2012/034472).

Fungicides Suitable as Mixing Partners are:

(1) Ergosterol biosynthesis inhibitors, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifin, nuarimol, oxpoconazole, paclobutrazole, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-[1-(4-methoxyphenoxy)-3,3-dimethylbutan-2-yl] 1H-imidazole-1-carbothioate.

(2) Respiration inhibitors (respiratory chain inhibitors), for example bixafen, boscalid, carboxin, diflumetorim, fenfuram, fluopyram, flutolanil, fluxapyroxad, furametpyr, furmecyclox, isopyrazam mixture of the syn-epimeric racemate 1RS,4SR,9RS and of the anti-epimeric racemate 1RS,4SR,9SR, isopyrazam (anti-epimeric racemate), isopyrazam (anti-epimeric enantiomer 1R,4S,9S), isopyrazam (anti-epimeric enantiomer 1S,4R,9R), isopyrazam (syn-epimeric racemate 1RS,4SR,9RS), isopyrazam (syn-epimeric enantiomer 1R,4S,9R), isopyrazam (syn-epimeric enantiomer 1S,4R,9S), mepronil, oxycarboxin, penflufen, penthiopyrad, sedaxane, thifluzamid, 1-methyl-N-[2-(1,1,2,2-tetrafluoroethoxy)phenyl]-3-(trifluoromethyl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, 5,8-difluoro-N-[2-(2-fluoro-4-{[4-(trifluoromethyl)pyridin-2-yl]oxy]phenyl)ethyl]quinazoline-4-amine, N-[9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.

(3) Respiration inhibitors (respiratory chain inhibitors) acting on complex III of the respiratory chain, for example ametoctradin, amisulbrom, azoxystrobin, cyazofamid, coumethoxystrobin, coumoxystrobin, dimoxystrobin, enestroburin, famoxadone, fenamidone, fenoxystrobin, fluoxastrobin, kresoxim-methyl, metominostrobin, orysastrobin, picoxystrobin, pyraclostrobin, pyrametostrobin, pyraoxystrobin, pyribencarb, triclopyricarb, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, methyl (2E)-2-{2-[({cyclopropyl[(4-methoxyphenyl)imino]methyl}sulfanyl)methyl]phenyl}-3-methoxyprop-2-enoate, N-(3-ethyl-3,5,5-trimethylcyclohexyl)-3-(formylamino)-2-hydroxybenzamide, 2-{2-[(2,5-dimethylphenoxy)methyl]phenyl}-2-methoxy-N-methylacetamide and (2R)-2-{2-[(2,5-dimethylphenoxy)methyl]phenyl}-2-methoxy-N-methylacetamide.

(4) Mitosis and cell division inhibitors, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)[1,2,4]triazolo[1,5-a]pyrimidine and 3-chloro-5-(6-chloropyridin-3-yl)-6-methyl-4-(2,4,6-trifluorophenyl)pyridazine.

(5) Compounds with multisite activity, for example Bordeaux mixture, captafol, captan, chlorothalonil, copper preparations such as copper hydroxide, copper naphthenate, copper oxide, copper oxychloride, copper sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulfur and sulfur preparations, for example calcium polysulfide, thiram, tolylfluanid, zineb and ziram.

(6) Resistance inductors, for example acibenzolar-S-methyl, isotianil, probenazole and tiadinil.

(7) Amino acid and protein biosynthesis inhibitors, for example andoprim, blasticidin-S, cyprodinil, kasugamycin, kasugamycin hydrochloride hydrate, mepanipyrim, pyrimethanil and 3-(5-fluoro-3,3,4,4-tetramethyl-3,4-dihydroisoquinolin-1-yl)quinoline.

(8) Inhibitors of ATP production, for example, fentin acetate, fentin chloride, fentin hydroxide and silthiofam.

(9) Cell wall synthesis inhibitors, for example benthiavalicarb, dimethomorph, flumorph, iprovalicarb, mandipropamid, polyoxins, polyoxorim, validamycin A and valifenalate.

(10) Lipid and membrane synthesis inhibitors, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.

(11) Melanin biosynthesis inhibitors, for example carpropamid, diclocymet, fenoxanil, fthalide, pyroquilon, tricyclazole and 2,2,2-trifluoroethyl {3-methyl-1-[(4-methylbenzoyl)amino]butan-2-yl}carbamate.

(12) Nucleic acid synthesis inhibitors, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.

(13) Signal transduction inhibitors, for example chlozolinate, fenpiclonil, fludioxonil, iprodione, procymidone, quinoxyfen and vinclozolin.

(14) Decouplers, for example binapacryl, dinocap, ferimzone, fluazinam and meptyldinocap.

(15) Further compounds, for example benthiazole, bethoxazin, capsimycin, carvone, chinomethionat, pyriofenone (chlazafenone), cufraneb, cyflufenamid, cymoxanil, cyprosulfamide, dazomet, debacarb, dichlorophen, diclomezine, difenzoquat, difenzoquat methylsulfate, diphenylamine, ecomat, fenpyrazamine, flumetover, fluoromide, flusulfamide, flutianil, fosetyl-aluminum, fosetyl-calcium, fosetyl-sodium, hexachlorobenzene, irumamycin, methasulfocarb, methyl isothiocyanate, metrafenon, mildiomycin, natamycin, nickel dimethyldithiocarbamate, nitrothal-isopropyl, octhilinone, oxamocarb, oxyfenthiin, pentachlorophenol and salts thereof, phenothrin, phosphoric acid and salts thereof, propamocarb-fosetylate, propanosine-sodium, proquinazid, pyrimorph, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one, pyrrolnitrin, tebufloquin, tecloftalam, tolnifanid, triazoxide, trichlamide, zarilamide, (3S,6S,7R, 8R)-8-benzyl-3-[({3-[(isobutyryloxy)methoxy]-4-methoxypyridin-2-yl}carbony)amino]-6-methyl-4,9-dioxo-1,5-dioxonan-7-yl 2-methylpropanoate, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2,6-dimethyl-1H,5H-[1,4]dithiino[2,3-c:5,6-c′]dipyrrole-1,3,5,7(2H,6H)-tetrone, 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]pyridine, 2-phenylphenol and salts thereof, 3-(4,4,5-trifluoro-3,3-dimethyl-3,4-dihydroisoquinolin-1-yl)quinoline, 3,4,5-trichloropyridine-2,6-dicarbonitrile, 3-[5(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]pyrimidine-4-amine, 5-fluoro-2-[(4-methylbenzyl)oxy]pyrimidine-4-amine, 5-methyl-6-octyl[1,2,4]triazolo[1,5-a]pyrimidine-7-amine, ethyl (2Z)-3-amino-2-cyano-3-phenylprop-2-enoate, N′-(4-{[3-(4-chlorobenzyl)-1,2,4-thiadiazol-5-yl]oxy}-2,5-dimethylphenyl)-N-ethyl-N-methylimidoformamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(E)-[(cyclopropylmethoxy)imino][6-(difluoromethoxy)-2,3-difluorophenyl]methyl}-2-phenylacetamide, N—{(Z)-[(cyclopropylmethoxy)imino][6-(difluoromethoxy)-2,3-difluorophenyl]methyl}-2-phenylacetamide, N′-{4-[(3-tert-butyl-4-cyano-1,2-thiazol-5-yl)oxy]-2-chloro-5-methylphenyl}-N-ethyl-N-methylimidoformamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, pentyl {6-[({[(1-methyl-1H-tetrazol-5-yl)(phenyl)methylidene]amino}oxy)methyl]pyridin-2-yl}carbamate, phenazine-1-carboxylic acid, quinolin-8-ol, quinolin-8-ol sulfate (2:1) and tert-butyl {6-[({[(1-methyl-1H-tetrazol-5-yl)(phenyl)methylene]amino}oxy)methyl]pyridin-2-yl}carbamate.

(16) Further compounds, for example 1-methyl-3-(trifluoromethyl)-N-[2′-(trifluoromethyl)biphenyl-2-yl]-1H-pyrazole-4-carboxamide, N-(4′-chlorobiphenyl-2-yl)-3-(difluoromethyl)-1-methyl-1H-pyrazole-4-carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1 -yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N-(4′-ethynylbiphenyl-2-yl)-5-fluoro-1,3-dimethyl-1H-pyrazole-4-carboxamide, 2-chloro-N-(4′-ethynylbiphenyl-2-yl)pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1H-pyrazole-4-carboxamide, 5-fluoro-N-[4′-(3-methoxy-3-methylbut-1-yn-1-yl)biphenyl-2-yl]-1,3-dimethyl-1H-pyrazole-4-carboxamide, 2-chloro-N-[4′-(3-methoxy-3-methylbut-1-yn-1-yl)biphenyl-2-yl]pyridine-3-carboxamide, (5-bromo-2-methoxy-4-methylpyridin-3-yl)(2,3,4-trimethoxy-6-methylphenyl)methanone, N-[2-(4-{[3-(4-chlorophenyl)prop-2-yn-1-yl]oxy}-3-methoxyphenyl)ethyl]-N2-(methylsulfonyl)valinamide, 4-oxo-4-[(2-phenylethyl)amino]butanoic acid and but-3-yn-1-yl {6-[({[(Z)-(1-methyl-1H-tetrazol-5-yl)(phenyl)methylene]amino}oxy)methyl]pyridin-2-yl }carbamate.

All the mixing partners mentioned in classes (1) to (16), as the case may be, may form salts with suitable bases or acids if they are capable of doing so on the basis of their functional groups.

All plants and plant parts can be treated in accordance with the invention. Plants are understood here to mean all plants and plant populations such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested material and vegetative and generative propagation material, for example cuttings, tubers, rhizomes, slips and seeds.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been grown by conventional breeding, by mutagenesis or by recombinant DNA techniques. They may be cultivars, varieties, biotypes or genotypes.

The treatment of the plants and plant parts with the active compounds, active compound combinations or compositions according to the invention or the active compounds, active compound combinations or compositions to be used in accordance with the invention is carried out directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore by dry seed treatment, wet seed treatment, slurry treatment, incrusting, coating with one or more coats, etc. It is furthermore possible to apply the active compounds by the ultra-low volume method or to inject the active compound preparation or the active compound itself into the soil.

One preferred direct treatment of the plants is foliar application, meaning that the active compounds, active compound combinations or compositions are applied to the foliage, where the frequency of treatment and the application rate may be adjusted for the infestation pressure of the particular pathogen, pest or weed.

In the case of systemically active compounds, the active compounds, active compound combinations or compositions access the plants via the root system. The treatment of the plants then proceeds through the action of the active compounds, active compound combinations or compositions on the habitat of the plant. This can be done, for example, by drenching, or by mixing into the soil or the nutrient solution, meaning that the locus of the plant (e.g. soil or hydroponic systems) is impregnated with a liquid form of the active compounds, active compound combinations or compositions, or by soil application, i.e. the active compounds, active compound combinations or compositions according to the invention are introduced in solid form (e.g. in the form of granules) into the locus of the plants. In the case of paddy rice crops, this can also be done by metering the invention in a solid application form (for example as granules) into a flooded paddy field.

The control of animal pests by the treatment of the seed of plants has long been known and is the subject of constant improvements. Nevertheless, the treatment of seed gives rise to a series of problems which cannot always be solved in a satisfactory manner Thus, it is desirable to develop methods for protecting the seed and the germinating plant which dispense with, or at least reduce considerably, the additional application of crop protection compositions during storage, after sowing or after emergence of the plants. It is additionally desirable to optimize the amount of active compound used so as to provide optimum protection for the seed and the germinating plant from attack by animal pests, but without damage to the plant itself by the active compound used. More particularly, methods for the treatment of seed should also take account of the intrinsic insecticidal or nematicidal properties of pest-resistant or pest-tolerant transgenic plants, in order to achieve optimum protection of the seed and of the germinating plant with minimum expenditure of crop protection products.

The present invention therefore also relates particularly to a method for protection of seed and germinating plants from attack by pests, by treating the seed with an active compound according to the invention or an active compound to be used in accordance with the invention. The method of the invention for protection of seed and germinating plants against attack by pests comprises a method in which the seed is treated simultaneously in one operation with an active compound of the formula I and a mixing partner. It also comprises a method where the seed is treated at different times with an active compound of the formula I and a mixing partner.

The invention likewise relates to the use of the active compounds according to the invention for the treatment of seed for protecting the seed and the resulting plant from animal pests.

Furthermore, the invention relates to seed which has been treated with an active compound according to the invention for protection from animal pests. The invention also relates to seed which has been treated simultaneously with an active compound of the formula I and a mixing partner. The invention further relates to seed which has been treated at different times with an active compound of the formula I and a mixing partner. In the case of seed which has been treated at different times with an active compound of the formula I and a mixing partner, the individual active compounds in the composition according to the invention may be present on the seed in different layers. In this case, the layers comprising an active compound of the formula I and a mixing partner may optionally be separated by an intermediate layer. The invention also relates to seed where an active compound of the formula I and a mixing partner have been applied as a component of a coating or as a further layer or further layers in addition to a coating.

The invention further relates to seed which, after the treatment with the active compound of the formula (I) or an active compound combination comprising the active compound of the formula (I), has been subjected to a film-coating process to prevent dust abrasion on the seed.

One of the advantages of the present invention is that the particular systemic properties of the compositions according to the invention mean that treatment of the seed with these compositions protects not only the seed itself but also the resulting plants after emergence from animal pests. In this way, the immediate treatment of the crop at the time of sowing or shortly thereafter can be dispensed with.

A further advantage is considered to be that the treatment of the seed with active compound of the formula (I) or active compound combination comprising the active compound of the formula (I) can promote germination and emergence of the treated seed.

It is likewise to be considered to be advantageous that active compounds of the formula (I) and the active compound combinations mentioned can also be used for transgenic seed in particular.

It should also be mentioned that active compounds of the formula (I) can be used in combination with signalling technology compositions, which results, for example, in better colonization by symbionts, for example rhizobia, mycorrhizae and/or endophytic bacteria, and/or leads to optimized nitrogen fixation.

The compositions according to the invention are suitable for protection of seed of any plant variety which is used in agriculture, in the greenhouse, in forests or in horticulture. More particularly, this is the seed of cereals (for example wheat, barley, rye, millet and oats), corn, cotton, soya beans, rice, potatoes, sunflowers, coffee, tobacco, canola, oilseed rape, beets (for example sugar beets and fodder beets), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), corn, soya bean, cotton, canola, oilseed rape and rice.

As already mentioned above, the treatment of transgenic seed with active compounds of the formula (I) or an active compound combination is also of particular significance. This involves the seed of plants which generally contain at least one heterologous gene which controls the expression of a polypeptide having insecticidal and/or nematicidal properties in particular. In this context, the heterologous genes in transgenic seed may be derived from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

Within the context of the present invention, the active compound of the formula (I) is applied to the seed alone (or as an active compound combination) or in a suitable formulation. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition according to the invention and/or further additives applied to the seed is chosen such that the germination of the seed is not impaired and the plant which arises therefrom is not damaged. This has to be ensured particularly in the case of active compounds which can exhibit phytotoxic effects at certain application rates.

The compositions according to the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compounds/active compound combinations which can be used in accordance with the invention can be converted to the customary seed-dressing formulations, such as solutions, emulsions, suspensions, powders, foams, slurries or other coating compositions for seed, and also ULV formulations.

These formulations are prepared in a known manner, by mixing the active compounds/active compound combinations with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active compounds. Preference is given to using alkyl naphthalenesulfonates, such as diisopropyl or diisobutyl naphthalenesulfonates.

Suitable dispersants and/or emulsifiers which may be present in the seed-dressing formulations usable in accordance with the invention are all nonionic, anionic and cationic dispersants customary for the formulation of active agrochemical compounds. Preference is given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulfated derivatives thereof. Suitable anionic dispersants are especially lignosulfonates, polyacrylic acid salts and arylsulfonate/formaldehyde condensates.

Antifoams which may be present in the seed-dressing formulations usable in accordance with the invention are all foam-inhibiting substances customary for formulation of agrochemically active compounds. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz-und Schädlingsbekämpfungsmittel”, vol. 2, Springer Verlag, 1970, pp. 401-412).

The seed-dressing formulations usable in accordance with the invention can be used to treat a wide variety of different kinds of seed, either directly or after prior dilution with water. Thus, the concentrates or the preparations obtainable therefrom by dilution with water may be used to dress the seed of cereals, such as wheat, barley, rye, oats, and triticale, and also the seed of corn, rice, oilseed rape, peas, beans, cotton, sunflowers, and beets, or else vegetable seed of any of a very wide variety of kinds. The seed-dressing formulations usable in accordance with the invention, or the dilute preparations thereof, can also be used to dress seed of transgenic plants. In this case, additional synergistic effects may also occur in interaction with the substances formed by expression.

For treatment of seed with the seed-dressing formulations usable in accordance with the invention, or the preparations prepared therefrom by adding water, all mixing units usable customarily for the seed dressing are useful. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The application rate of the seed-dressing formulations usable in accordance with the invention can be varied within a relatively wide range. It depends on the particular content $ of the active compound(s) in the formulations and on the seed. The application rates in the case of $ active compounds/active compound combinations are generally between 0.001 and 50 g per kilogram of seed, preferably between 0.01 and 15 g per kilogram of seed.

The prior art does not disclose whether the active compounds of the formula (I) are effective against biotic stress factors and/or abiotic stress of plants or with respect to plant growth.

It has now been found that the active compounds of the formula (I) according to the invention are suitable for enhancing the defences of the plant (pathogen control in plants).

It is known that plants react to natural stress conditions such as, for example, cold temperatures, heat, drought, injury, attack by pathogens (viruses, bacteria, fungi), insects etc., but also to herbicides, with specific or unspecific defence mechanisms (Pflanzenbiochemie [Plant Biochemistry], pp. 393-462, Spektrum Akademischer Verlag, Heidelberg, Berlin, Oxford, Hans W. Heldt, 1996.; Biochemistry and Molecular Biology of Plants, pp. 1102-1203, American Society of Plant Physiologists, Rockville, Md., eds. Buchanan, Gruissem, Jones, 2000). In this case, for example, cell wall components formed through injury or specific signal substances originating from the pathogen serve as inductors of plant signal transduction chains which ultimately lead to the formation of defensive molecules directed against the stress factor. These may be, for example, (a) low molecular weight substances, for example phytoalexins, (b) non-enzymatic proteins such as pathogenesis-related proteins (PR proteins), (c) enzymatic proteins such as chitinases, glucanases, or (d) specific inhibitors of essentiel proteins such as protease inhibitors, xylanase inhibitors, which attack the pathogen directly or hinder its proliferation (Dangl and Jones, Nature 411, 826-833, 2001; Kessler and Baldwin, Annual Review of Plant Biology, 53, 299-328, 2003).

An additional defence mechanism it the so-called hypersensitive reaction (HR) which is mediated via oxidative stress and causes death of plant tissue in the region of the centre of an infection, thus preventing the spread of plant pathogens which are dependent on living cells (Pennazio, New Microbiol. 18, 229-240, 1995).

Later in the progression of an infection, the plant's messenger substances transmit signals to unaffected tissues, which leads to triggering of defence reactions in these tissues too and prevents secondary infections (Systemic acquired resistance, SAR) (Ryals et al., The Plant Cell 8, 1809-1819, 1996).

A number of signaling substances which are endogenous to plants and are involved in stress tolerance or pathogenic defence are already known. Examples here include salicylic acid, benzoic acid, jasmonic acid or ethylene (Biochemistry and Molecular Biology of Plants, pp. 850-929, American Society of Plant Physiologists, Rockville, Maryland, eds. Buchanan, Gruissem, Jones, 2000). Some of these substances or the stable synthetic derivatives and derived structures thereof are also effective on external application to plants or in seed dressing, and activate defence reactions which cause elevated stress tolerance or pathogen tolerance of the plant (Sembdner, Parthier, Ann. Rev. Plant Physiol. Plant Mol. Biol. 44, 569-589, 1993). Salicylate-mediated defence is directed particularly against phytopathogenic fungi, bacteria and viruses (Ryals et al., The Plant Cell 8, 1809-1819, 1996).

A known synthetic product which has a function similar to that of salicylic acid and can mediate a protective effect against phytopathogenic fungi, bacteria and viruses is benzothiadiazole (CGA 245704; common name: acibenzolar-S-methyl; trade name: Bion®) (Achuo et al., Plant Pathology 53 (1), 65-72, 2004; Tamblyn et al., Pesticide Science 55 (6), 676-677, 1999; EP-OS [European Published Specification] 0 313 512).

Other compounds which belong to the group of the oxylipins, for example jasmonic acid, and the protective mechanisms they trigger are particularly effective against harmful insects (Walling, J. Plant Growth Regul. 19, 195-216, 2000).

It is additionally known that treatment of plants with insecticides from the group of the neonicotinoids (chloronicotinyls) leads to increased resistance of the plant to abiotic stress. This is especially true of imidacloprid (Brown et al., Beltwide Cotton Conference Proceedings 2231-2237, 2004). This protection results from modification of physiological and biochemical properties of the plant cells, for example improvement of membrane stability, increasing the carbohydrate concentration, increasing the polyol concentration and antioxidant activity (Gonias et al., Beltwide Cotton Conference Proceedings 2225-2229, 2004).

Also known is the effect of chloronicotinyls on biotic stress factors (Crop Protection 19 (5), 349-354, 2000; Journal of Entomological Science 37(1), 101-112, 2002; Annals of Biology (Hisar, India) 19 (2), 179-181, 2003). For example, insecticides from the group of the neonicotinoids (chloronicotinyls) lead to increased expression of genes from the group of the pathogenesis-related proteins (PR proteins). PR proteins support the plants primarily in defence against biotic stressors, for example phytopathogenic fungi, bacteria and viruses (DE 10 2005 045 174 A; DE 10 2005 022 994 A and WO 2006/122662 A; Thielert Pflanzenschutz-Nachrichten Bayer, 59 (1), 73-86, 2006; Francis et al., European Journal of Plant Pathology, publ. online 23, Jan. 2009).

It is additionally known that treatment of genetically modified plants with insecticides from the group of the neonicotinoids (chloronicotinyls) leads to improved stress tolerance of the plant (EP 1 731 037 A), for example with respect to the herbicide glyphosate too (WO 2006/015697 A).

It is thus known that plants possess several endogenous reaction mechanisms which can bring about effective defence against a wide variety of harmful organisms (biotic stress) and/or abiotic stress.

The growing of healthy young plants with uniform growth forms an essential prerequisite for cultivation of large areas and economical crop management of agricultural, horticultural and forestry crop plants.

Numerous methods for growing young plants are established in agriculture, forestry and horticulture. In this context, the growing substrates used, as well as steamed soil, are also special substrates based, inter alia, on peat mosses, coconut fibres, rockwool such as Grodan®, pumice, expanded clay such as Lecaton® or Lecadan®, clay granules such as Seramis®, foams such as Baystrat®, vermiculite, perlite, synthetic soil such as Hygromull®, or combinations of these substrates, into which seed, either undressed or dressed with fungicides and/or insecticides, is sown.

In specific crops such as tobacco, young plants are increasingly reared by the float method or floating method (Leal, R. S., The use of Confidor S in the float, a new tobacco seedlings production system in the South of Brazil. Pflanzenschutz-Nachrichten Bayer (German edition) (2001), 54(3), pages 337 to 352; Rudolph, R. D.; Rogers, W. D.; The efficacy of imidacloprid treatment for reduction in the severity of insect vectored virus diseases of tobacco. Pflanzenschutz-Nachrichten Bayer (German edition) (2001), 54(3), pages 311 to 336). In this method, the seed is sown in special containers, for example Styropor tablets with holes, in special growing soil based on peat culture substrate, and then cultivated in containers with suitable nutrient solution until the desired transplantation size is reached (FIG. 1). The containers are allowed to float on the nutrient solution, from which the name of the growing method derives (Leal, 2001, see above). In floating methods, sucking pests have been controlled for a number of years using insecticides from the class of the neonicotinoids (chloronicotinyls). Typically, the plants in the float method are sprayed with neonicotinoid (chloronicotinyls) insecticides shortly before transplantation, or they are watered with neonicotinoid (chloronicotinyls) insecticides immediately before or during transplantation, which is referred to as drenching (Leal, 2001, see above; Rudolph and Rogers, 2001, see above). Both application methods are technically relatively complex.

To protect the emerging seed or planting stock from fungal pathogens and pests, fungicides and insecticides are used here until transplantation. The choice of crop protection compositions, the location and the timing of the application and the application rate of the compositions depend here particularly on the kind of fungal diseases and pests that occur, on the specific mode of action and duration of action of the compositions and on their compatibility with plants, and can therefore be adapted directly to the specific requirements of different crops and regions.

Irrespective of any insect control, the active compounds of the formula (I) lead to good protection of the plants from damage by fungal, bacterial or viral pathogens.

Without wishing to be tied to a theory, it is currently assumed that the defence against the pathogens results from the induction of PR proteins as a consequence of treatment with at least one active compound of the formula (I).

More particularly, the use according to the invention shows the advantages described in the treatment of seed, in soil treatment, in specific growing and cultivation methods (for example floating box, rockwool, hydroponic), but also in stem and foliar treatment. Combinations of an active compound of the formula (I) with insecticides, fungicides and bactericides, inter alia, show synergistic action in the control of plant diseases. The combined use of the active compounds of the formula (I) with cultivars genetically modified with a view to increased abiotic stress tolerance additionally leads to a synergistic improvement in growth.

Finally, it has also been found in accordance with the invention that the active compounds of the formula (I) are suitable not only for enhancing pathogen defence in plants but also for improving plant growth and/or for enhancing the resistance of plants to plant diseases caused by fungi, bacteria, viruses, MLO (Mycoplasma-like organisms) and/or RLO (Rickettsia-like organisms), especially to soil-borne fungal diseases, and/or for increasing the resistance of plants to abiotic stress factors.

Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or lack of shade.

The present invention therefore firstly provides for the use of at least one active compound of the formula (I) for enhancing the defences of plants and/or for improving plant growth and/or for enhancing the resistance of plants to plant diseases caused by fungi, bacteria, viruses, MLO (Mycoplasma-like organisms) and/or RLO (Rickettsia-like organisms), especially to soil-borne fungal diseases, and/or for enhancing the resistance of plants to abiotic stress factors.

In the context of the present invention, the term “plant growth” is understood to mean various benefits for plants that are not directly associated with the known pesticidal activity, preferably insecticidal activity, of the active compounds of the formula (I). Such beneficial properties are, for example, the following improved plant characteristics: accelerated germination and emergence of seed and planting stock, improved root growth with regard to surface area and depth, increased stolon or tiller formation, stronger and more productive stolons and tillers, improvement in shoot growth, increased lodging resistance, increased shoot base diameter, increased leaf area, greener leaf colour, higher yields of nutrients and constituents, for example carbohydrates, fats, oils, proteins, vitamins, minerals, essential oils, dyes, fibres, better fibre quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soils and water, enhanced tolerance to UV radiation, enhanced tolerance to ozone stress, improved compatibility with respect to herbicides and other plant treatment compositions, improved water absorption and photosynthesis performance, advantageous plant properties, for example acceleration of ripening, more homogeneous ripening, greater attractiveness to beneficial animals, improved pollination, or other advantages well known to a person skilled in the art.

The further various benefits for plants mentioned above can be combined in a known manner in component form, and generally applicable terms can be used to describe them. Such terms are, for example, the following names: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms with which a person skilled in the art is entirely familiar.

It has also been found that active compounds of the formula (I) lead to increased expression of genes from the group of the pathogenesis-related proteins (PR proteins). PR proteins support the plants primarily in the defence against biotic stressors, for example phytopathogenic fungi, bacteria and viruses. The result of this is that plants, after application of active compounds of the formula (I), are better protected against infections by phytopathogenic fungi, bacteria and viruses. In the event that it is necessary to use insecticides, fungicides and bactericides in a mixture with active compounds of the formula (I), including in sequential application, the action of the latter is promoted.

It has additionally been found in accordance with the invention that the application of the active compounds of the formula (I) in combination with a fertilizer as defined below to plants or in the environment thereof has a synergistic growth-enhancing effect.

Fertilizers which can be used in accordance with the invention together with the active compounds or compositions which have been elucidated in detail above are generally organic and inorganic nitrogen compounds, for example ureas, urea/formaldehyde condensates, amino acids, ammonium salts and ammonium nitrates, potassium salts (preferably chlorides, sulfates, nitrates), salts of phosphoric acid and/or salts of phosphorous acid (preferably potassium salts and ammonium salts). In this context, particular mention should be made of the NPK fertilizers, i.e. fertilizers which contain nitrogen, phosphorus and potassium, calcium ammonium nitrate, i.e. fertilizers which additionally contain calcium, or ammonium sulfate nitrate (general formula (NH₄)₂SO₄ NH₄NO₃), ammonium phosphate and ammonium sulfate. These fertilizers are generally known to the person skilled in the art; see also, for example, Ullmann's Encyclopedia of Industrial Chemistry, 5th edition, Vol. A 10, pages 323 to 431, Verlagsgesellschaft, Weinheim, 1987.

The fertilizers may also contain salts of micronutrients (preferably calcium, sulfur, boron, manganese, magnesium, iron, boron, copper, zinc, molybdenum and cobalt) and phytohormones (for example vitamin B1 and indol-3-yl acetic acid (IAA)) or mixtures thereof. Fertilizers used in accordance with the invention may also contain other salts such as monoammonium phosphate (MAP), diammonium phosphate (DAP), potassium sulfate, potassium chloride or magnesium sulfate. Suitable amounts for the secondary nutrients or trace elements are amounts of 0.5% to 5% by weight, based on the overall fertilizer. Further possible constituents are crop protection agents, insecticides or fungicides, growth regulators or mixtures thereof. Further details of these are given further down.

The fertilizers can be used, for example, in the form of powders, granules, prills or compactates. However, the fertilizers can also be used in liquid form, dissolved in an aqueous medium. In this case, dilute aqueous ammonia can also be used as a nitrogen fertilizer. Further possible ingredients for fertilizers are described, for example, in Ullmann's Encyclopedia of Industrial Chemistry, 5th edition, 1987, volume A 10, pages 363 to 401, DE-A 41 28 828, DE-A 19 05 834 and DE-A 196 31 764.

The general composition of the fertilizers, which, in the context of the present invention, may take the form of straight and/or compound fertilizers, for example composed of nitrogen, potassium or phosphorus, may vary within a wide range. In general, a content of 1% to 30% by weight of nitrogen (preferably 5% to 20% by weight), of 1% to 20% by weight of potassium (preferably 3% to 15% by weight) and a content of 1% to 20% by weight of phosphorus (preferably 3% to 10% by weight) is advantageous. The content of microelements is usually in the ppm range, preferably in the range from 1 to 1000 ppm.

In the context of the present invention, the fertilizer and the active compound of the formula (I) can be administered simultaneously, i.e. synchronously. However, it is also possible first to apply the fertilizer and then the active compound of the formula (I), or first to apply the active compound of the formula (I) and then the fertilizer. In the case of nonsynchronous application of the active compound of the formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, the active compounds of the general formula (I) according to the invention and the fertilizer are applied within a time frame of less than 1 hour, preferably less than 30 minutes, more preferably less than 15 minutes.

In addition, it is possible to produce dimensionally stable mixtures, for example in the form of rods, granules, tablets etc., proceeding from at least one active compound for use in accordance with the invention and at least one fertilizer. In order to produce a corresponding dimensionally stable mixture, the appropriate components can be mixed with one another and optionally extruded, or the at least one active compound of the formula (I) for use in accordance with the invention can be applied to the fertilizer. If appropriate, it is also possible to use formulation auxiliaries in the dimensionally stable mixtures, for example extenders or adhesives, to achieve dimensional stability of the resulting mixture. By virtue of the corresponding dimensional stability, corresponding mixtures are particularly suitable for use in the home & garden sector, i.e. for a domestic user or amateur gardener, who is able to use the dimensionally stable mixture or the components thereof in a predetermined, clearly defined amount and without any particular aids.

Irrespective of this, the mixtures comprising at least one of the active compounds for use in accordance with the invention and the at least one fertilizer may also be in liquid form, such that—for example in the case of a professional user in the field of agriculture—the resulting mixture may be deployed as a tankmix.

Through the use of at least one of the active compounds for use in accordance with the invention and at least one fertilizer, it is possible to achieve increased root growth which, in turn, enables higher nutrient uptake and hence promotes plant growth.

The active compounds for use in accordance with the invention, optionally in combination with fertilizers, can preferably be employed in the following plants, although the enumeration which follows is not limiting.

Preferred plants are those from the group of the useful plants, ornamental plants, turfgrass types, commonly used trees which are employed as ornamentals in public and domestic areas, and forestry trees. Forestry trees include trees for the production of timber, cellulose, paper and products made from parts of the trees.

The term useful plants as used here refers to crop plants which are used as plants for obtaining foods, animal feeds, fuels or for industrial purposes.

The useful plants include, for example, the following types of plants: turf, vines, cereals, for example wheat, barley, rye, oats, triticale, rice, corn and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soya beans; oil crops, for example oilseed rape, mustard, poppies, olives, sunflowers, coconuts, castor oil plants, cacao beans and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, aubergine, sugar cane, tea, pepper, grapevines, hops, bananas, latex plants and ornamentals, for example flowers, shrubs, deciduous trees and coniferous trees such as conifers. This enumeration does not represent any limitation.

Particularly suitable target crops are considered to be the following plants: cotton, aubergine, turf, pome fruit, stone fruit, soft fruit, corn, wheat, barley, cucumber, tobacco, vines, rice, cereals, pear, beans, soya beans, oilseed rape, tomato, bell pepper, melons, cabbage, potatoes and apples.

Examples of trees include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees include: from the tree species Pinus: P. radiate, P. ponderosa, P. contorta, P. sylvestre, P. strobes; from the tree species Eucalyptus: E. grandis, E. globulus, E. camadentis.

Very particularly preferred trees include: horse chestnut, Platanaceae, linden tree, maple tree.

The present invention can also be applied to any desired turfgrasses, including cool-season turfgrasses and warm-season turfgrasses. Examples of cool-season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turfgrasses are Bermudagrass (Cynodon spp. L. C. Rich), zoysiagrass (Zoysia spp. Willd.), St. Augustine grass (Stenotaphrum secundatum Walt Kuntze), centipedegrass (Eremochloa ophiuroides Munro Hack.), carpetgrass (Axonopus affinis Chase), Bahia grass (Paspalum notatum Flugge), Kikuyugrass (Pennisetum clandestinum Hochst. ex Chiov.), buffalo grass (Buchloe dactyloids (Nutt.) Engelm.), Blue gramma (Bouteloua gracilis (H.B.K.) Lag. ex Griffiths), seashore paspalum (Paspalum vaginatum Swartz) and sideoats grama (Bouteloua curtipendula (Michx. Torr.)). Cool-season turfgrasses are generally preferred for the use according to the invention. Particular preference is given to bluegrass, bentgrass and redtop, fescues and ryegrasses. Bentgrass is especially preferred.

The active compounds of the formula (I) and compositions thereof are suitable for controlling animal pests in the hygiene sector. More particularly, the invention can be used in the domestic sector, in the hygiene sector and in the protection of stored products, particularly for control of insects, arachnids and mites encountered in enclosed spaces, for example dwellings, factory halls, offices, vehicle cabins. For controlling animal pests, the active compounds or compositions are used alone or in combination with other active compounds and/or auxiliaries. They are preferably used in domestic insecticide products. The active compounds according to the invention are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

In addition, the active compounds of the formula (I) can be used to control a multitude of different pests, including, for example, harmful sucking insects, biting insects and other pests which are plant parasites, stored material pests, pests which destroy industrial material, and hygiene pests including parasites in the animal health sector, and for the control thereof, for example the elimination and eradication thereof. The present invention thus also includes a method for controlling pests.

In the animal health field, i.e. in the field of veterinary medicine, the active compounds according to the invention are active against animal parasites, especially ectoparasites or endoparasites. The term “endoparasites” includes especially helminths and protozoa, such as coccidia. Ectoparasites are typically and preferably arthropods, especially insects and acarids.

In the field of veterinary medicine, the compounds according to the invention having favourable homeotherm toxicity are suitable for the control of parasites encountered in animal breeding and animal husbandry in livestock, breeding, zoo, laboratory, experimental and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, even insects such as bees.

Domestic animals include, for example, mammals, such as hamsters, guinea pigs, rats, mice, chinchillas, ferrets or especially dogs, cats; cage birds; reptiles; amphibians or aquarium fish.

In a preferred embodiment, the compounds according to the invention are administered to mammals

In another preferred embodiment, the compounds according to the invention are administered to birds, namely cage birds or especially poultry.

The use of the active compounds according to the invention for the control of animal parasites is intended to reduce or prevent illness, cases of deaths and performance losses (in the case of meat, milk, wool, hides, eggs, honey and the like), so that more economical and simpler animal keeping is enabled and better animal well-being is achievable.

In relation to the field of animal health, the term “control” or “controlling” means that the active compounds can effectively reduce the incidence of the respective parasite in an animal infected with such parasites to a harmless degree. More specifically, “controlling” as used herein means that the active compound can kill the respective parasite, inhibit its growth, or inhibit its proliferation.

Examples of arthropods include, but without any limitation:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp.,Werneckiella spp., Lepikentron spp., Damalina spp., Trichodectes spp., Felicola spp.; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order of the Siphonapterida, for example Pulex spp., Ctenocephalides spp., Tunga spp., Xenopsylla spp., Ceratophyllus spp.; from the order of the Heteropterida, for example Cimex spp., Triatoma spp., Rhodnius spp., Panstrongylus spp.; as well as nuisance and hygiene pests from the order of the Blattarida.

In addition, among the arthropods, examples of Acari include the following, but without any limitation:

from the subclass of the Acari (Acarina) and the order of the Metastigmata, for example from the family of Argasidae like Argas spp., Ornithodorus spp., Otobius spp., from the family of Ixodidae like Ixodes spp., Amblyomma spp., Rhipicephalus (Boophilus) spp., Dermacentor spp., Haemophysalis spp., Hyalomma spp., Rhipicephalus spp. (the original genus of multi-host ticks); from the order of Mesostigmata like Dermanyssus spp., Ornithonyssus spp., Pneumonyssus spp., Raillietia spp., Pneumonyssus spp., Sternostoma spp., Varroa spp., Acarapis spp.; from the order of the Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Examples of parasitic protozoa include, but without any limitation:

Mastigophora (Flagellata), for example Trypanosomatidae, for example Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, for example Trichomonadidae, for example Giardia lamblia, G. canis.

Sarcomastigophora (Rhizopoda) such as Entamoebidae, for example Entamoeba histolytica, Hartmanellidae, for example Acanthamoeba sp., Harmanella sp.

Apicomplexa (Sporozoa) such as Eimeridae, for example, Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example Toxoplasma gondii, Hammondia heydornii, Neospora caninum, Besnoitia besnoitii; such as Sarcocystidae, for example Sarcocystis bovicanis, S. bovihominis, S. ovicanis, S. ovifelis, S. neurona, S. spec., S. suihominis, such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Examples of pathogenic endoparasites, which are helminths, include platyhelmintha (e.g. monogenea, cestodes and trematodes), roundworms, acanthocephala, and pentastoma. Further helminths include, but without any limitation:

Monogenea: for example: Gyrodactylus spp., Dactylogyrus spp., Polystoma spp.

Cestodes: from the order of the Pseudophyllidea for example: Diphyllobothrium spp., Spirometra spp., Schistocephalus spp., Ligula spp., Bothridium spp., Diplogonoporus spp.

From the order of the Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: from the class of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.

Roundworms: Trichinellida, for example: Trichuris spp., Capillaria spp., Trichomosoides spp., Trichinella spp.

From the order of the Tylenchida, for example: Micronema spp., Strongyloides spp.

From the order of the Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp.

Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.

From the order of the Echinorhynchida, for example, Acanthocephalus spp., Echinorhynchus spp., Leptorhynchoides spp.

Pentastoma: from the order of the Porocephalida, for example, Linguatula spp.

In the veterinary field and in animal keeping, the active compounds according to the invention are administered by methods commonly known in the art, such as enterally, parenterally, dermally or nasally, in the form of suitable preparations. Administration may be prophylactic or therapeutic.

Thus, one embodiment of the present invention refers to compounds according to the invention for use as a medicament.

A further aspect relates to compounds according to the invention for use as an antiendoparasitic agent, in particular a helminthicidal agent or antiprotozoic agent. For example, compounds according to the invention are suitable for use as an antiendoparasitic agent, in particular a helminthicidal agent or antiprotozoic agent, for example in animal husbandry, in animal keeping, in animal housing and in the hygiene sector.

Yet a further aspect relates to compounds according to the invention for use as an antiectoparasitic agent, in particular an arthropodicidal agent, such as an insecticide or acaricide. For example, compounds according to the invention are suitable for use as an antiectoparasitic agent, especially an arthropodicidal agent such as an insecticide or acaricide, for example in animal keeping, in animal husbandry, in animal housing and in the hygiene sector.

The active compounds of the formula (I) and compositions comprising them are suitable for protection of industrial materials against attack or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In one embodiment of the invention, the compositions or products according to the invention also comprise at least one further insecticide and/or at least one fungicide.

In a further embodiment, this composition according to the invention is a ready-to-use composition, meaning that it can be applied to the appropriate material without any further modifications. Useful further insecticides or fungicides include those mentioned above.

It has also been found that, surprisingly, the active compounds and compositions according to the invention can be used to protect objects which come into contact with saltwater or brackish water, especially hulls, screens, nets, buildings, moorings and signalling systems, against fouling. The active compounds and compositions according to the invention can again be used alone or in combinations with other active compounds as antifouling compositions.

### PREPARATION EXAMPLES

The preparation and use examples which follow illustrate the invention without limiting it. The products were characterized by 1H NMR spectroscopy and/or LC-MS (liquid chromatography-mass spectrometry) and/or GC-MS (gas chromatography-mass spectrometry).

The log P values were determined analogously to OECD Guideline 117 (EC Directive 92/69/EEC) by HPLC (high-performance liquid chromatography) using reversed-phase columns (C 18), by the following methods:

[a] The LC-MS determination in the acidic range was effected at pH 2.7 with 0.1% aqueous formic acid and acetonitrile (contains 0.1% formic acid) as eluents; linear gradient from 10% acetonitrile to 95% acetonitrile. log P^{[a]} is also referred to as log P(HCOOH).

[b] LC-MS determination in the neutral range was effected at pH 7.8 with 0.001 molar aqueous ammonium hydrogencarbonate solution and acetonitrile as eluents; linear gradient from 10% acetonitrile to 95% acetonitrile. log P^{[b]} is also referred to as log P(neutral).

Calibration is effected with solutions of a homologous series of unbranched alkan-2-ones (having 3 to 16 carbon atoms) with known log P values (log P values determined on the basis of the retention times by linear interpolation between two successive alkanones).

The NMR spectra were measured with a Bruker II Avance 400 equipped with a 1.7 mm TCI probe head. In isolated cases, the NMR spectra were determined using a Bruker Avance II 600.

The NMR data for selected examples are listed in conventional form (δ values, multiplet splitting, number of hydrogen atoms). The splitting of the signals was described as follows: s (singlet), d (doublet), t (triplet), q (quartet), m (multiplet), broad (for broad signals). Solvents used were CD₃CN, CDCl₃ or D6-DMSO, and tetramethylsilane (0.00 ppm) was used as reference.

Alternatively, the NMR data of selected examples can be reported as NMR peak lists.

NMR peak list method

When the 1H NMR data for selected examples are noted in the form of 1H NMR peak lists, first the δ value in ppm and then the signal intensity is listed for each signal peak, separated by a space. The δ value—signal intensity—number pairs for different signal peaks are listed with separation from one another by semicolons.

The peak list for one example therefore takes the form of:

δ₁ intensity₁;δ₂ intensity₂; . . . ;δᵢ intensityᵢ; . . . ; δₙ intensityₙ.

The solvent in which the NMR spectrum was recorded is listed in square brackets after the number of the example and before the NMR peak list or the conventional NMR interpretation list.

The GC-MS spectra are determined using an Agilent 6890 GC, HP 5973 MSD on a dimethylsilicone phase, using a temperature gradient from 50° C. to 320° C. GC-MS indices are determined as Kovats indices using solutions of a homologous series of n-alkanes (having an even number of 8 to 38 carbon atoms).

### Preparation Example 1

### 3-{2-Fluoro-4-methyl-5-[(2,2,2-trifluoroethyl)sulfinyl]phenyl}pyrido[2,3-d]pyrimidin-4(3H)-one (Ex. No. 5)

### Step 1: 2-Amino-N-{2-fluoro-4-methyl-5-[(2,2,2-trifluoroethyl)sulfanyl]phenyl}nicotinamide

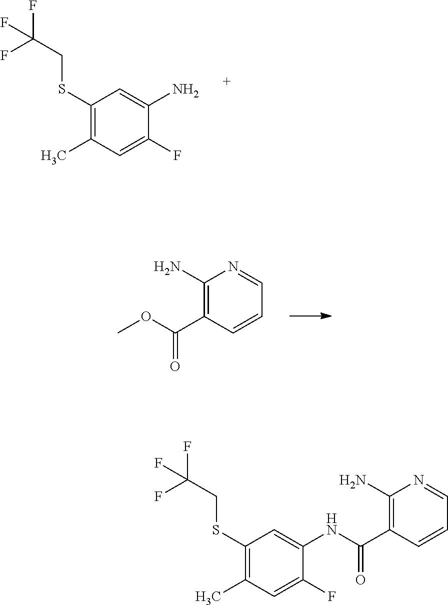 [SMILES: Cc1cc(F)c(N)cc1SCC(F)(F)F ; COC(=O)c1cccnc1N ; Cc1cc(F)c(NC(=O)c2cccnc2N)cc1SCC(F)(F)F]

500 mg (2.09 mmol) of 2-fluoro-4-methyl-5-[(2,2,2-trifluoroethyl)sulfanyl]aniline were initially charged in 10 ml of 1,2-dichloroethane. 2.09 ml of a 2M solution of trimethylaluminum in toluene were added dropwise, and the reaction mixture was stirred at room temperature for 30 min 318 mg (2.09 mmol) of methyl 2-aminopyridine-3-carboxylate were added and the mixture was heated under reflux overnight. After cooling, the mixture was diluted with water and dilute hydrochloric acid and extracted with methylene chloride. The organic phase was washed with water, dried over sodium sulfate, filtered and freed from the solvent under reduced pressure. The residue was purified by column chromatography using MPLC on RP(C-18) with water, formic acid (1 ml/l) and acetonitrile (30-60%). This gave 290 mg of the title compound (37% of theory, purity 96% according to LC/MS).

1H-NMR(D6-DMSO) δ ppm: 10.03(s,1H), 8.16-8.13(m,2H), 8.09(dd,1H), 7.71(d,1H), 7.27(d,1H), 7.07(bs,2H), 3.87(q,2H), 2.41(s,3H)

log P (HCOOH): 1.70 log P(neutral): 2.71

### Step 2: 3-{2-Fluoro-4-methyl-5-[(2,2,2-trifluoroethyl)sulfanyl]phenyl}-2-pyrido [2,3-d]pyrimidin-4(3H)-one (Ex. No. 2)

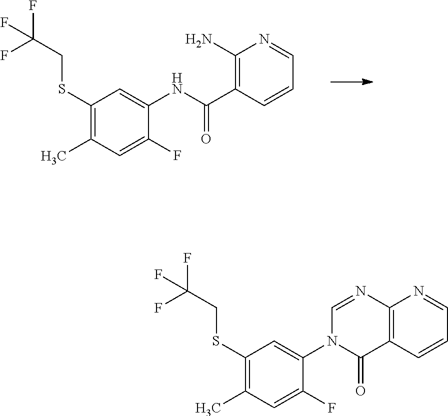 [SMILES: Cc1cc(F)c(NC(=O)c2cccnc2N)cc1SCC(F)(F)F ; Cc1cc(F)c(-n2cnc3ncccc3c2=O)cc1SCC(F)(F)F]

100 mg (0.28 mmol) of 2-amino-N-{2-fluoro-4-methyl-5-[(2,2,2-trifluoroethyl)sulfanyl]phenyl}nicotinamide were initially charged in 0.37 ml (2.80 mmol) of N,N-dimethylformamide dimethyl acetal, and the mixture was stirred at 100° C. overnight. After cooling, the reaction mixture was concentrated and the residue was taken up in 1 ml of formic acid and heated under reflux for 2 hours. After cooling and removal of the solvent, the reaction mixture was partitioned between a saturated sodium bicarbonate solution and methylene chloride. The organic phase was washed with water, dried over sodium sulfate, filtered and freed from the solvent under reduced pressure. The residue was purified by column chromatography using MPLC on RP(C-18) with water, formic acid (1 ml/l) and acetonitrile (40-70%). This gave 41 mg of the title compound (40% of theory, purity 100% according to LC/MS).

1H-NMR(D6-DMSO) δ ppm: 9.05(dd,1H), 8.63-8.50(m,2H), 7.91(d,1H), 7.66(dd,1H), 7.49(d,1H), 4.01(q,2H), 2.46(s,3H)

log P (HCOOH): 2.43 log P(neutral): 2.43

### Step 3: 3-{2-Fluoro-4-methyl-5-[(2,2,2-trifluoroethyl)sulfinyl]phenyl}pyrido[2,3-d]pyrimidin-4(3H)-one (Ex. No. 5)

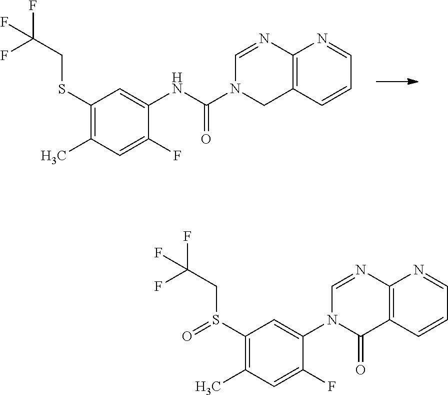 [SMILES: Cc1cc(F)c(NC(=O)N2C=Nc3ncccc3C2)cc1SCC(F)(F)F ; Cc1cc(F)c(-n2cnc3ncccc3c2=O)cc1S(=O)CC(F)(F)F]

35 mg (0,10 mmol) of 3-{2-fluoro-4-methyl-5-[(2,2,2-trifluoroethyl)sulfanyl]phenyl}pyrido[2,3-d]pyrimidin-4(3H)-one were initially charged in methyl chloride, 18 mg (0.10 mmol) of meta-chloroperbenzoic acid were added and the reaction mixture was stirred at room temperature overnight and then extracted with sodium thiosulfate and sodium bicarbonate solution. The organic phase was dried over sodium sulfate and filtered. Removal of the solvent under reduced pressure gave 36 mg of the title compound (94% of theory, purity 96% according to LC/MS).

1H-NMR(D6-DMSO) δ ppm: 9.05(dd,1H), 8.65(s,1H), 8.61(dd,1H), 8.17(d,1H), 7.67(dd,1H), 7.61(d,1H), 4.30-4.03(m,2H), 3H under the DMSO peak

log P (HCOOH): 1.56 log P(neutral): 1.56

### Preparation Example 2

### 3-{2-Fluoro-4-methyl-5-[(2,2,2-trifluoroethyl)sulfinyl]phenyl}-2-mehtylpyrido[2,3-d]pyrimidin-4(3H)-one (Ex. No. 10)

### Step 1: 2-Methyl-4H-pyrido[2,3-d][1,3]oxazin-4-one

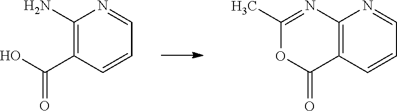

5 g (36.1 mmol) of 2-aminonicotinic acid are heated under reflux in acetic anhydride for 1 h. Cooling and removal of the solvent under reduced pressure gives 5.9 g of product (100% of theory, purity 100% according to LC/MS).

1H-NMR(D6-DMSO) δ ppm: 8.97-8.95(m,1H), 8.51-8.48(m,1H), 7.62-7.59(m,1H), 2.45(s,3H)

log P (HCOOH): 0.42 log P(neutral): 0.43

### Step 2: 3-{2-Fluoro-4-methyl-5-[(2,2,2-trifluoroethyl)sulfanyl]phenyl}-2-mehtylpyrido[2,3-d]pyrimidin-4(3H)-one (Ex. No. 8)

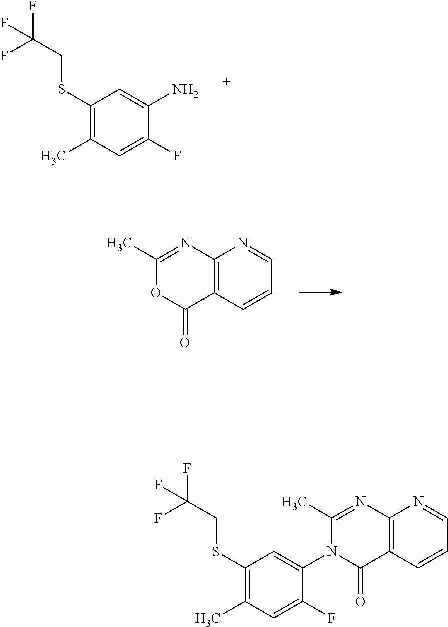 [SMILES: Cc1cc(F)c(N)cc1SCC(F)(F)F ; Cc1nc2ncccc2c(=O)o1 ; Cc1cc(F)c(-n2c(C)nc3ncccc3c2=O)cc1SCC(F)(F)F]

200 mg (0.83 mmol) of 2-fluoro-4-methyl-5-[(2,2,2-trifluoroethyl)sulfanyl]aniline and 136 mg (0.83 mmol) of 2-methyl-4H-pyrido[2,3-d][1,3]oxazin-4-one are heated under reflux in ethanol (10 ml) for 30 min. The filtrate is concentrated and taken up in diglyme (10 ml), and 136 mg (0.83 mmol) of 2-methyl-4H-pyrido[2,3-d][1,3]oxazin-4-one were added. The reaction mixture was stirred at 120° C. for 5 h and the solvent was then removed under reduced pressure. The residue was purified by column chromatography using MPLC on RP(C-18) with water, formic acid (1 ml/l) and acetonitrile (40-70%). This gives 10 mg of product (3% of theory, purity 100% according to LC/MS).

1H-NMR(D6-DMSO) δ ppm: 9.02-9.01(m,1H), 8.54-8.51(m,1H), 7.90(d,1H), 7.60-7.57(m,1H), 7.51(d,1H), 4.06-3.93(m,2H), 2.46(s,3H), 2.24(s,3H)

log P (HCOOH): 2.55 log P(neutral): 2.52

### Step 3: 3-{2-Fluoro-4-methyl-5-[(2,2,2-trifluoroethyl)sulfinyl]phenyl}-2-mehtylpyrido[2,3-d]pyrimidin-4(3H)-one (Ex. No. 10)

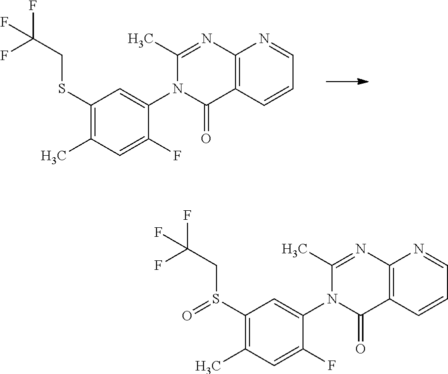 [SMILES: Cc1cc(F)c(-n2c(C)nc3ncccc3c2=O)cc1SCC(F)(F)F ; Cc1cc(F)c(-n2c(C)nc3ncccc3c2=O)cc1S(=O)CC(F)(F)F]

14 mg (0,03 mmol) of 3-{2-fluoro-4-methyl-5-[(2,2,2-trifluoroethyl)sulfanyl]phenyl}-2-mehtylpyrido[2,3-d]pyrimidin-4(3H)-one are initially charged in methyl chloride, 9 mg (0.04 mmol) of meta-chloroperbenzoic acid were added and the reaction mixture was stirred at room temperature for 15 h. The mixture is extracted with sodium thiosulfate solution and sodium bicarbonate solution. The organic phase is dried over sodium sulfate and filtered, and the solvent was removed under reduced pressure. The residue was purified by column chromatography using MPLC with cyclohexane and acetone (0-30%). This gives 6 mg of product (36% of theory, purity 95% according to LC/MS).

1H-NMR(D6-DMSO) δ ppm: 9.02-9.00(m,1H), 8.53-8.51(m,1H), 8.18-8.15(m,1H), 7.66-7.62(m,1H), 7.60-7.57(m,1H), 4.39-3.91(m,2H), 2.31(s,3H), 2.23(s,3H)

log P(HCOOH): 1.66 log P(neutral): 1.64

By the above-described processes, the following compounds of the general formula (I) were prepared:

TABLE 1

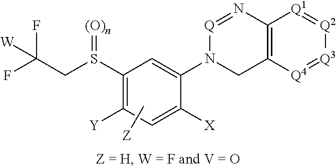

| Compound | | | | | | | | |
|---|---|---|---|---|---|---|---|---|
| number | n | Y | X | Q | Q¹ | Q² | Q³ | Q⁴ |
| 1 | 0 | CH₃ | F | CH | CH | CH | N | CH |
| 2 | 0 | CH₃ | F | CH | N | CH | CH | CH |
| 3 | 0 | Cl | Cl | CH | N | CH | CH | CH |
| 4 | 0 | CH₃ | CH₃ | CH | N | CH | CH | CH |
| 5 | 1 | CH₃ | F | CH | N | CH | CH | CH |
| 6 | 1 | CH₃ | CH₃ | CH | N | CH | CH | CH |
| 7 | 1 | Cl | Cl | CH | N | CH | CH | CH |
| 8 | 0 | CH₃ | F | C—CH₃ | N | CH | CH | CH |
| 9 | 0 | CH₃ | F | CH | CH | CH | CH | N |
| 10 | 1 | CH₃ | F | C—CH₃ | N | CH | CH | CH |
| 11 | 1 | CH₃ | F | CH | CH | CH | CH | N |
| 12 | 0 | CH₃ | F | CH | CH | N | CH | CH |
| 13 | 1 | CH₃ | F | CH | CH | N | CH | CH |
| 14 | 1 | CH₃ | F | CH | CH | CH | N | CH |
| 15 | 0 | CH₃ | Cl | CH | N | CH | CH | CH |
| 16 | 0 | CH₃ | CH₃ | C—CH₃ | N | CH | CH | CH |
| 17 | 1 | CH₃ | CH₃ | C—CH₃ | N | CH | CH | CH |
| 18 | 1 | CH₃ | Cl | CH | N | CH | CH | CH |

log P data of the compounds according to Table 1:

| Compound | | |
|---|---|---|
| number | logP(HCOOH) | logP(neutral) |
| 1 | 2.54 | 2.56 |
| 2 | 2.43 | 2.43 |
| 3 | 2.73 | 2.68 |
| 4 | 2.53 | 2.54 |
| 5 | 1.56 | 1.56 |
| 6 | 1.62 | 1.61 |
| 7 | 1.98 | 1.84 |
| 8 | 2.55 | 2.52 |
| 9 | 2.33 | |
| 10 | 1.66 | 1.64 |
| 11 | 1.49 | 1.46 |
| 12 | 2.55 | 2.56 |
| 13 | 1.60 | 1.63 |
| 14 | 1.61 | 1.64 |
| 15 | 2.62 | 2.62 |
| 16 | 2.63 | 2.60 |
| 17 | 1.67 | 1.67 |
| 18 | 1.77 | 1.73 |

NMR data of the compounds according to Table 1:
Compound No. 1, solvent: [DMSO], spectrometer: 399.95 MHz
9.1755 (4.77); 9.1741 (4.71); 8.7943 (4.27); 8.7813 (4.49); 8.524 (7.01); 8.0744 (3.18); 8.0724
(3.12); 8.0614 (3.05); 8.0595 (3); 7.9185 (2.97); 7.9002 (2.99); 7.5109 (2.51); 7.4838 (2.48); 4.0467
(1.21); 4.0209 (3.83); 3.9951 (4.01); 3.9693 (1.39); 3.3329 (17.12); 2.6721 (0.38); 2.5254 (1.27);
2.5119 (20.74); 2.5075 (41.31); 2.503 (54.67); 2.4984 (40.89); 2.4941 (20.59); 2.4652 (16); 2.3297
(0.38); 2.0767 (1.36); 0.008 (0.98); −0.0002 (28.56); -0.0085 (1.2)
Compound No. 2, solvent: [DMSO], spectrometer: 399.95 MHz
9.0579 (1.9); 9.053 (2.05); 9.0464 (2.01); 9.0416 (1.97); 8.6308 (2.13); 8.6259 (2.18); 8.611 (2.44);
8.602 (6.96); 7.92 (2.89); 7.9018 (2.86); 7.6787 (2.17); 7.6672 (2.11); 7.6589 (2.08); 7.6474 (2.05);
7.5018 (2.51); 7.4748 (2.49); 5.756 (0.32); 4.0481 (1.2); 4.0225 (3.73); 3.9967 (3.88); 3.9708 (1.32);
3.3216 (103.72); 2.6751 (0.65); 2.6705 (0.91); 2.6662 (0.66); 2.5235 (2.62); 2.5059 (97.76); 2.5015
(129.97); 2.497 (97.46); 2.4931 (49.27); 2.4637 (16); 2.3328 (0.65); 2.3283 (0.88); 2.3237 (0.68); −0.0002
(0.89)
Compound No. 3, solvent: [DMSO], spectrometer: 399.95 MHz
10.4958 (0.32); 9.0757 (4.04); 9.0707 (4.48); 9.0642 (4.57); 9.0592 (4.4); 8.6476 (4.59); 8.6427
(4.7); 8.634 (0.72); 8.6279 (5.2); 8.6229 (4.77); 8.6145 (0.48); 8.5721 (16); 8.5546 (0.45); 8.5412
(0.92); 8.3151 (2.44); 8.1518 (0.45); 8.1171 (10.62); 8.0728 (14.45); 8.0146 (0.88); 7.9773 (0.37);
7.8482 (0.56); 7.8201 (0.87); 7.6938 (4.33); 7.6823 (4.27); 7.674 (4.24); 7.6625 (4.3); 5.7554 (1.51);
4.2435 (0.44); 4.2294 (0.89); 4.2186 (1.23); 4.2043 (2.77); 4.1936 (2.82); 4.179 (2.86); 4.1682
(2.82); 4.1538 (1.43); 4.1427 (1.11); 4.1287 (0.53); 4.102 (0.58); 4.0925 (1.09); 3.6785 (1.57);
3.6623 (0.71); 3.648 (2.97); 3.4343 (0.32); 3.4139 (0.37); 3.3936 (0.37); 3.3211 (865.23); 3.2551
(0.35); 3.2374 (0.96); 3.142 (0.48); 3.0511 (0.66); 2.7413 (0.35); 2.7069 (0.56); 2.697 (0.64); 2.6797
(2.37); 2.6751 (4.76); 2.6705 (6.46); 2.6659 (4.65); 2.6614 (2.13); 2.642 (0.36); 2.6261 (0.39);
2.5407 (2.91); 2.5239 (18.4); 2.5191 (28.61); 2.5105 (344.48); 2.506 (687.71); 2.5014 (909.85);
2.4968 (653.76); 2.4923 (304.05); 2.3373 (2.08); 2.3328 (4.45); 2.3282 (6.11); 2.3236 (4.35); 2.319
(1.93); 1.9886 (0.43); 1.2589 (0.44); 1.2343 (2.52); 1.1477 (0.69); 0.8543 (0.33); 0.1459 (2.52);
0.008 (21.78); −0.0002 (650.21); −0.0086 (19.78); −0.0199 (0.73); −0.0257 (0.52); −0.0287 (0.46); −0.0956
(0.36); −0.1497 (2.52)
Compound No. 4, solvent: [DMSO], spectrometer: 399.95 MHz
9.0468 (1.84); 9.0419 (1.97); 9.0354 (1.97); 9.0306 (1.81); 8.6247 (2.41); 8.6197 (2.36); 8.605 (2.6);
8.6 (2.36); 8.4655 (8); 7.6616 (7.94); 7.6503 (2.33); 7.642 (2.18); 7.6305 (2.14); 7.3477 (4.63);
5.7561 (0.82); 4.0243 (1.17); 3.9985 (3.58); 3.9726 (3.67); 3.9466 (1.25); 3.3223 (125.53); 2.6751
(0.92); 2.6706 (1.19); 2.6662 (0.87); 2.559 (0.34); 2.5237 (3.95); 2.506 (131.72); 2.5015 (168.4);
2.4971 (123.85); 2.493 (60.89); 2.4016 (15.47); 2.3737 (0.45); 2.3645 (0.35); 2.3328 (0.96); 2.3283
(1.23); 2.3239 (0.94); 2.1915 (0.36); 2.0711 (16); 1.9886 (0.45); 1.2339 (1.08); −0.0002 (1.22)
Compound No. 5, solvent: [DMSO], spectrometer: 399.95 MHz
20.0117 (0.33); 9.0562 (4.89); 9.0513 (5.7); 9.0448 (5.59); 9.0398 (5.8); 8.6522 (16); 8.6302 (6.11);
8.6252 (6.49); 8.618 (1.07); 8.6105 (6.93); 8.6054 (7.23); 8.3154 (1.57); 8.182 (7.25); 8.1635 (7.47);
7.6793 (5.92); 7.6678 (5.64); 7.6595 (5.74); 7.648 (5.83); 7.6199 (5.03); 7.593 (5.09); 7.1975 (0.34);
7.1755 (0.38); 6.7718 (0.45); 6.7493 (0.39); 5.7559 (4.94); 4.9692 (0.36); 4.292 (0.91); 4.256 (1.4);
4.2283 (1.3); 4.2025 (0.47); 4.1215 (0.97); 4.0946 (1.13); 4.0578 (0.82); 3.3225 (437.21); 2.7604
(1.45); 2.6796 (1.87); 2.6751 (3.97); 2.6705 (5.52); 2.666 (3.98); 2.6613 (1.86); 2.524 (14.83);
2.5192 (22.17); 2.5106 (287.39); 2.506 (585.61); 2.5014 (785.18); 2.4968 (574.82); 2.4923 (291.48);
2.3374 (1.77); 2.3328 (3.96); 2.3283 (5.48); 2.3236 (3.8); 2.3191 (1.67); 1.2346 (1.63); 1.1745
(0.45); 1.1481 (0.68); 0.1459 (0.35); 0.008 (3.2); −0.0001 (93.72); −0.0085 (2.2)
Compound No. 6, solvent: [DMSO], spectrometer: 399.95 MHz
9.0504 (1.25); 9.0454 (2.93); 9.0402 (2.38); 9.0339 (3.04); 9.0288 (1.87); 8.6262 (1.91); 8.6214
(2.9); 8.6167 (1.34); 8.6065 (2.11); 8.6016 (3); 8.5969 (1.31); 8.5328 (6.75); 8.5151 (4.54); 8.3153
(1.33); 7.9272 (4.48); 7.9216 (3.1); 7.6686 (1.21); 7.6628 (1.92); 7.6571 (1.29); 7.6513 (1.87); 7.649
(1.49); 7.6431 (1.77); 7.6373 (1.27); 7.6317 (1.75); 7.4829 (2.01); 7.4577 (2.93); 5.7559 (0.88);
4.3101 (0.4); 4.2825 (0.5); 4.2734 (0.52); 4.2456 (0.51); 4.1706 (0.44); 4.1438 (1.32); 4.1387 (1.25);
4.1168 (1.35); 4.1116 (1.39); 4.0898 (0.47); 4.0846 (0.49); 3.9602 (0.5); 3.9332 (0.59); 3.9236
(0.47); 3.8967 (0.46); 3.3226 (415.12); 2.6795 (1.46); 2.675 (3.13); 2.6705 (4.36); 2.6659 (3.13);
2.6612 (1.46); 2.5239 (11.5); 2.5192 (17.86); 2.5105 (231.71); 2.506 (471.43); 2.5013 (631.16);
2.4967 (459.46); 2.4922 (218.04); 2.4475 (7.97); 2.4405 (10.83); 2.4097 (0.48); 2.4015 (0.56); 2.357
(0.45); 2.3373 (1.52); 2.3327 (3.25); 2.3281 (4.43); 2.3236 (3.12); 2.319 (1.49); 2.2863 (0.44);
2.1751 (16); 1.234 (1.37); 1.1471 (0.55); 0.0081 (2.05); −0.0001 (68.91); −0.0085 (2.07)
Compound No. 7, solvent: [DMSO], spectrometer: 399.95 MHz
10.6285 (0.9); 9.0608 (3.5); 9.0529 (3.56); 8.653 (0.7); 8.6483 (0.68); 8.6374 (3.74); 8.6324 (4.18);
8.6255 (3.16); 8.6178 (3.76); 8.6125 (4.08); 8.6109 (4.02); 8.6058 (2.94); 8.5827 (9.98); 8.5422
(12.34); 8.4957 (0.55); 8.484 (0.55); 8.3518 (0.9); 8.3339 (1.39); 8.3157 (3.45); 8.2973 (16); 8.2857
(11.02); 8.2763 (11); 8.201 (0.58); 8.1967 (0.62); 8.1822 (0.67); 8.1773 (0.63); 8.1414 (0.4); 8.0936
(0.39); 8.015 (1.79); 7.6854 (4.16); 7.674 (4.06); 7.6655 (3.99); 7.6541 (3.79); 7.5263 (0.53); 7.514
(0.61); 7.5067 (0.51); 7.4951 (0.53); 7.3428 (0.47); 7.3334 (0.51); 5.756 (5.26); 4.5648 (0.33);
4.5378 (1.12); 4.527 (0.46); 4.5106 (1.29); 4.5004 (1.4); 4.484 (0.49); 4.4737 (1.43); 4.4465 (0.49);
4.3533 (0.52); 4.3277 (1.24); 4.3184 (0.99); 4.3008 (1.59); 4.291 (2.23); 4.2739 (0.84); 4.2639
(2.38); 4.2357 (2.22); 4.2258 (0.5); 4.2084 (2.04); 4.1984 (1.05); 4.1815 (0.87); 4.1714 (1.15);
4.1427 (1.58); 4.1361 (0.6); 4.1324 (0.66); 4.1159 (1.83); 4.1055 (1.55); 4.0898 (0.88); 4.0788
(1.55); 4.0613 (0.34); 4.0523 (0.74); 3.4993 (0.47); 3.3949 (0.55); 3.3876 (0.54); 3.3811 (0.68);
3.3565 (1.18); 3.3209 (883.53); 3.2439 (2.72); 3.2229 (0.33); 3.1468 (0.37); 3.048 (0.72); 3.0414
(0.68); 2.6795 (3.56); 2.675 (7.31); 2.6705 (10.14); 2.6659 (7.24); 2.6614 (3.46); 2.6031 (0.55);
2.5972 (0.64); 2.5831 (0.92); 2.5407 (5.24); 2.5238 (30.92); 2.5104 (553.33); 2.506 (1095.22);
2.5014 (1450.63); 2.4968 (1060.46); 2.4923 (509.28); 2.4196 (0.76); 2.3657 (0.49); 2.3507 (0.43);
2.3372 (3.42); 2.3327 (7.05); 2.3282 (9.68); 2.3236 (7); 2.3194 (3.21); 1.3513 (0.47); 1.234 (2.02);
1.1493 (2.7); 0.8542 (0.33); 0.1461 (3.12); 0.008 (28.59); −0.0002 (779.13); −0.0085 (26.2); −0.0432
(0.41); −0.0482 (0.4); −0.1496 (3.2)
Compound No. 8, solvent: [DMSO], spectrometer: 399.95 MHz
9.0217 (1.92); 9.0167 (1.95); 9.0103 (1.93); 9.0052 (1.76); 8.5377 (1.86); 8.5328 (1.82); 8.518
(2.03); 8.5131 (1.91); 8.316 (2.14); 7.9141 (2.29); 7.8957 (2.25); 7.6032 (1.85); 7.5916 (1.74);
7.5836 (1.72); 7.5719 (1.8); 7.5223 (2.05); 7.4957 (2.03); 4.0642 (0.43); 4.0499 (0.53); 4.0384
(0.53); 4.0247 (1.4); 3.999 (1.52); 3.994 (1.42); 3.9677 (1.35); 3.9552 (0.46); 3.9422 (0.52); 3.928
(0.47); 3.3209 (261.03); 2.675 (4.87); 2.6705 (6.47); 2.666 (4.8); 2.6007 (0.49); 2.5407 (4.78);
2.5236 (21.76); 2.5102 (387.73); 2.5059 (769.41); 2.5015 (1005.41); 2.4969 (723.64); 2.4926
(349.22); 2.4644 (15); 2.3824 (0.46); 2.3326 (4.72); 2.3281 (6.49); 2.3237 (4.77); 2.2402 (16);
2.1933 (0.39); 2.1757 (0.75); 2.1587 (0.4); 1.9144 (0.4); 1.4734 (0.33); 1.3982 (0.75); 1.3505 (0.63);
1.3359 (0.44); 1.2983 (0.69); 1.2585 (1.54); 1.2356 (14.82); 0.8705 (0.54); 0.854 (1.63); 0.8364
(0.68); 0.1461 (1.46); 0.0079 (12.95); −0.0001 (342.43); −0.0083 (12.74); −0.1498 (1.52)
Compound No. 9, solvent: [DMSO], spectrometer: 399.95 MHz
8.8959 (2.36); 8.892 (2.51); 8.8851 (2.53); 8.8812 (2.47); 8.4603 (7.29); 8.3148 (0.77); 8.2231
(2.33); 8.2192 (2.41); 8.2023 (2.83); 8.1984 (2.68); 7.9307 (3.01); 7.9196 (5.25); 7.9099 (2.79);
7.8993 (4.77); 7.5008 (2.44); 7.4737 (2.4); 4.0489 (1.21); 4.023 (3.86); 3.9972 (4.01); 3.9714 (1.38);
3.3195 (144.34); 2.6794 (0.53); 2.6751 (1.11); 2.6705 (1.52); 2.666 (1.09); 2.6613 (0.54); 2.5408
(1.03); 2.5239 (4.22); 2.5191 (6.39); 2.5105 (81.31); 2.506 (166.15); 2.5014 (223.7); 2.4968
(163.69); 2.4923 (77.78); 2.466 (16); 2.3373 (0.5); 2.3328 (1.07); 2.3283 (1.49); 2.3236 (1.08);
2.3191 (0.49); 2.0858 (0.6); 1.398 (1.46); 1.2346 (0.57); 0.146 (0.61); 0.008 (5.02); −0.0002
(147.55); −0.0085 (4.56); −0.1496 (0.61)
Compound No. 10, solvent: [DMSO], spectrometer: 399.95 MHz
19.9656 (0.54); 13.111 (0.75); 12.0395 (0.54); 9.0212 (2.04); 9.0163 (3.27); 9.0095 (2.61); 9.0049
(3.11); 9.0005 (1.61); 8.5313 (2.59); 8.5263 (2.83); 8.5115 (2.88); 8.5064 (3.04); 8.3151 (5.07);
8.1798 (2.72); 8.1658 (1.91); 8.1606 (2.67); 8.148 (1.88); 7.6576 (2.14); 7.6491 (1.58); 7.6315
(1.78); 7.6216 (1.5); 7.6056 (1.96); 7.5991 (1.65); 7.5941 (1.97); 7.5863 (2.77); 7.5793 (1.47);
7.5741 (2.08); 7.5677 (1.62); 5.0024 (0.56); 4.3922 (0.94); 4.3652 (1.18); 4.3546 (0.93); 4.3275
(1.17); 4.2587 (1.17); 4.2487 (1.04); 4.232 (1.03); 4.2212 (0.96); 4.1943 (0.54); 3.9768 (1.1); 3.9502
(1.03); 3.9399 (0.77); 3.9127 (0.92); 3.3193 (693.1); 2.769 (1.45); 2.6749 (7.26); 2.6704 (10.14);
2.6659 (7.46); 2.6612 (3.4); 2.6307 (0.73); 2.605 (0.83); 2.5843 (0.64); 2.5406 (6.29); 2.5237
(26.59); 2.5188 (43.17); 2.5104 (601.32); 2.5059 (1216.89); 2.5014 (1583.64); 2.4968 (1121.35);
2.4923 (531.49); 2.4077 (0.89); 2.4026 (0.82); 2.3858 (0.81); 2.3327 (7.21); 2.3282 (9.94); 2.3236
(7.55); 2.3192 (4.08); 2.3119 (10.25); 2.2856 (0.61); 2.2348 (2.44); 2.2275 (16); 2.1629 (0.65);
1.9762 (0.56); 1.2982 (0.72); 1.2587 (1.21); 1.2351 (3.4); 0.884 (0.86); 0.8679 (0.98); 0.146 (7.29);
0.0554 (0.64); 0.0433 (0.75); 0.036 (0.72); 0.0293 (1.52); 0.0079 (57.77); −0.0002 (1660.6); −0.0085
(54.08); −0.0247 (2.22); −0.0336 (1.23); −0.0504 (0.78); −0.059 (0.61); −0.121 (0.57); −0.1497 (7.36)
Compound No. 11, solvent: [DMSO], spectrometer: 399.95 MHz
20.0032 (0.57); 8.8938 (5.75); 8.8901 (6.68); 8.8831 (6.11); 8.879 (6.28); 8.5097 (16); 8.4634
(1.69); 8.3438 (0.7); 8.3244 (0.74); 8.315 (4.14); 8.2275 (5.71); 8.2236 (5.41); 8.2137 (1.04); 8.2066
(6.63); 8.2028 (6.57); 8.1965 (0.91); 8.1805 (7.13); 8.162 (7.13); 7.9291 (6.85); 7.9183 (6.47);
7.9084 (5.79); 7.8974 (5.87); 7.7814 (0.64); 7.6186 (5.38); 7.5925 (4.94); 4.9924 (0.65); 4.9706
(0.77); 4.3081 (0.6); 4.2564 (1.41); 4.2311 (1.24); 4.2012 (0.78); 4.0972 (1.25); 4.0869 (1.37); 4.069
(0.85); 4.0635 (0.91); 4.0565 (0.97); 4.041 (0.62); 3.3699 (0.73); 3.3529 (1.19); 3.3195 (895.35);
3.2994 (2.08); 3.2912 (1.38); 3.2825 (0.81); 2.7762 (0.67); 2.7623 (3.17); 2.6749 (7.8); 2.6703
(10.19); 2.6658 (7.32); 2.605 (0.74); 2.5814 (1.03); 2.5768 (1); 2.5404 (5.32); 2.5235 (29.55);
2.5102 (631.43); 2.5058 (1238.55); 2.5012 (1602.04); 2.4967 (1154.82); 2.4922 (538.48); 2.4478
(1.37); 2.4381 (1.54); 2.3855 (0.59); 2.3326 (7.73); 2.328 (10.32); 2.3234 (7.4); 2.2833 (0.9); 2.0857
(5.94); 1.3975 (3.77); 1.3359 (0.7); 1.2972 (0.82); 1.259 (1.42); 1.2519 (1.39); 1.235 (3.86); 1.1547
(0.6); 1.1398 (0.65); 0.8847 (1); 0.867 (1.22); 0.8542 (0.95); 0.8352 (0.66); 0.1459 (6.6); 0.0323
(0.81); 0.0078 (65.71); −0.0002 (1550.54); −0.0085 (52.37); −0.0202 (2.88); −0.029 (2.06); −0.0399
(1.45); −0.0604 (0.71); −0.0654 (0.83); −0.1498 (6.93)
Compound No. 12, solvent: [DMSO], spectrometer: 399.95 MHz
9.3823 (5.43); 9.3808 (5.41); 8.955 (4.89); 8.9486 (0.41); 8.941 (5.04); 8.6199 (0.35); 8.5996 (7.2);
8.3152 (0.7); 7.9219 (2.88); 7.9036 (2.9); 7.7164 (3.14); 7.715 (3.07); 7.7024 (3.04); 7.701 (2.97);
7.5054 (2.4); 7.4787 (2.37); 5.7556 (1.67); 4.0418 (1.23); 4.0163 (3.82); 3.9906 (3.97); 3.9647
(1.34); 3.3196 (127.97); 2.6749 (1.32); 2.6704 (1.83); 2.666 (1.33); 2.5552 (0.37); 2.5405 (1.28);
2.5237 (5.08); 2.5104 (108.93); 2.5059 (218.44); 2.5014 (285.39); 2.4968 (202.27); 2.4923 (94.66);
2.4799 (2.3); 2.4648 (16); 2.4277 (0.35); 2.3365 (1.31); 2.3327 (1.53); 2.3281 (1.85); 2.3235 (1.33);
2.0857 (0.91); 1.2355 (1.7); 0.1459 (1.24); 0.0079 (10.49); −0.0002 (292.18); −0.0085 (9.45); −0.0178
(0.37); −0.0222 (0.35); −0.1496 (1.27)
Compound No. 13, solvent: [DMSO], spectrometer: 399.95 MHz
9.3805 (12.53); 8.9535 (9.97); 8.9395 (10.25); 8.6443 (16); 8.3156 (0.85); 8.1893 (6.82); 8.1708
(6.82); 7.7201 (7.11); 7.7062 (6.87); 7.6221 (4.77); 7.5955 (4.75); 5.7558 (1.41); 4.2923 (0.78);
4.2574 (1.25); 4.2302 (1.09); 4.2034 (0.41); 4.107 (0.91); 4.0779 (1.14); 4.0441 (0.75); 3.3523
(0.37); 3.3208 (278.87); 3.2983 (0.52); 2.7605 (0.35); 2.6751 (1.77); 2.6706 (2.34); 2.6659 (1.62);
2.541 (1.02); 2.5238 (5.13); 2.5104 (133.37); 2.506 (273.77); 2.5015 (363.5); 2.4969 (264.62);
2.4925 (154.52); 2.4576 (0.88); 2.4373 (0.45); 2.3372 (0.91); 2.3328 (1.76); 2.3282 (2.52); 2.3234
(1.72); 1.2348 (0.8); −0.0003 (2.42)
Compound No. 14, solvent: [DMSO], spectrometer: 399.95 MHz
9.3804 (13.46); 8.9536 (10.61); 8.9396 (10.8); 8.7162 (0.42); 8.711 (0.46); 8.6448 (16); 8.5218
(0.79); 8.3158 (0.36); 8.1898 (7.37); 8.1714 (7.41); 8.1441 (0.38); 7.7977 (0.44); 7.7801 (0.4);
7.7201 (7.98); 7.7061 (7.75); 7.6223 (5.78); 7.5956 (5.75); 5.7561 (3.44); 4.3192 (0.32); 4.2933
(1.01); 4.2578 (1.56); 4.2305 (1.37); 4.2037 (0.5); 4.1326 (0.43); 4.1071 (1.19); 4.0791 (1.44);
4.0446 (0.96); 4.0169 (0.4); 3.3221 (119.3); 2.7789 (0.71); 2.6754 (0.96); 2.6709 (1.28); 2.6665
(0.95); 2.541 (0.75); 2.5062 (153.37); 2.5018 (198.84); 2.4973 (153.99); 2.4554 (1.2); 2.3924 (0.43);
2.3287 (1.52); 2.3242 (1.07); 1.2349 (0.9); 0.146 (0.4); 0.0078 (4.17); −0.0001 (91.54); −0.0083 (3.9);
−0.1495 (0.41)
Compound No. 15, solvent: [DMSO], spectrometer 400.0 MHz
9.065 (2.2); 9.060 (2.4); 9.053 (2.5); 9.049 (2.3); 9.041 (0.4); 8.913 (0.4); 8.890 (0.3); 8.637 (2.2);
8.632 (2.2); 8.623 (0.6); 8.617 (2.4); 8.612 (2.3); 8.603 (0.4); 8.546 (1.4); 8.537 (7.6); 7.940 (6.1);
7.688 (5.4); 7.683 (2.7); 7.671 (2.2); 7.663 (2.5); 7.651 (2.2); 7.643 (0.4); 7.587 (0.9); 7.573 (1.1);
4.113 (1.0); 4.087 (3.1); 4.062 (3.2); 4.036 (1.1); 3.326 (168.0); 3.198 (0.5); 2.898 (0.7); 2.882 (1.7);
2.800 (0.3); 2.794 (0.7); 2.675 (0.9); 2.671 (1.2); 2.666 (0.9); 2.629 (0.8); 2.541 (0.7); 2.524 (2.8);
2.506 (124.1); 2.502 (161.7); 2.497 (116.6); 2.476 (4.1); 2.426 (16.0); 2.333 (0.8); 2.328 (1.2); 2.321
(3.1); 2.117 (0.6); 1.398 (9.4); 1.234 (0.8); 1.140 (1.6); 0.008 (2.0); 0.000 (61.8); −0.008 (2.1)
Compound No. 16, solvent: [DMSO], spectrometer 400.0 MHz
9.006 (1.8); 9.001 (2.0); 8.995 (1.9); 8.990 (1.9); 8.530 (1.7); 8.525 (1.8); 8.510 (1.9); 8.505 (1.8);
8.142 (0.7); 7.650 (4.7); 7.579 (1.7); 7.568 (1.7); 7.560 (1.7); 7.548 (1.6); 7.359 (4.0); 5.757 (1.3);
4.064 (0.5); 4.050 (0.4); 4.039 (0.6); 4.025 (1.2); 4.014 (0.3); 4.000 (1.5); 3.974 (1.6); 3.948 (1.3);
3.935 (0.6); 3.922 (0.5); 3.909 (0.6); 3.324 (32.2); 3.238 (0.4); 2.671 (0.6); 2.506 (68.4); 2.502
(87.0); 2.498 (66.5); 2.397 (13.1); 2.329 (0.6); 2.162 (0.3); 2.143 (16.0); 2.086 (1.0); 2.007 (13.6);
1.398 (1.9); 1.236 (0.4); 0.000 (46.7)
Compound No. 17, solvent: [DMSO], spectrometer 400.0 MHz
9.008 (2.4); 9.004 (2.7); 8.997 (2.6); 8.992 (2.6); 8.526 (2.8); 8.521 (2.9); 8.506 (3.0); 8.501 (2.9);
8.316 (0.6); 7.902 (5.5); 7.896 (4.1); 7.587 (1.7); 7.582 (1.5); 7.575 (1.8); 7.567 (2.1); 7.562 (1.4);
7.556 (1.8); 7.551 (1.3); 7.499 (3.9); 7.488 (2.8); 4.315 (0.8); 4.306 (0.4); 4.288 (0.9); 4.278 (1.0);
4.260 (0.5); 4.251 (1.1); 4.243 (0.7); 4.216 (1.8); 4.189 (1.7); 4.162 (0.6); 3.940 (1.0); 3.930 (0.4);
3.913 (1.0); 3.903 (0.9); 3.886 (0.4); 3.876 (0.8); 3.849 (0.3); 3.387 (0.5); 3.365 (0.9); 3.328 (328.6);
3.296 (0.6); 2.712 (0.4); 2.671 (1.5); 2.506 (182.4); 2.502 (230.2); 2.498 (168.4); 2.457 (12.7); 2.441
(9.2); 2.328 (1.4); 2.221 (10.9); 2.128 (16.0); 2.113 (14.7); 2.108 (11.6); 1.297 (0.3); 1.258 (0.5);
1.235 (1.6); 1.140 (1.3); 0.854 (0.3); 0.146 (0.4); 0.007 (4.5); 0.000 (81.6); −0.149 (0.4)
Compound No. 18, solvent: [DMSO], spectrometer 400.0 MHz
9.057 (2.2); 9.053 (2.0); 9.046 (2.3); 9.042 (1.7); 8.632 (1.5); 8.627 (2.4); 8.622 (1.2); 8.612 (1.7);
8.607 (2.5); 8.603 (1.2); 8.573 (3.8); 8.556 (5.2); 8.536 (0.3); 8.315 (2.5); 8.194 (4.8); 8.188 (3.7);
7.857 (2.6); 7.831 (3.5); 7.682 (1.2); 7.678 (1.7); 7.671 (1.2); 7.666 (1.6); 7.658 (1.5); 7.651 (1.2);
7.647 (1.5); 4.414 (0.5); 4.387 (0.5); 4.377 (0.6); 4.350 (0.5); 4.253 (0.5); 4.244 (0.3); 4.226 (0.6);
4.216 (0.9); 4.189 (0.9); 4.162 (0.4); 4.142 (0.8); 4.114 (1.0); 4.105 (0.5); 4.086 (0.4); 4.078 (0.6);
4.037 (0.7); 4.010 (1.0); 3.983 (0.8); 3.973 (0.7); 3.946 (0.6); 3.852 (0.5); 3.507 (0.6); 3.323 (508.6);
2.671 (4.3); 2.667 (3.2); 2.506 (511.9); 2.502 (679.2); 2.497 (510.6); 2.378 (0.9); 2.328 (4.9); 2.283
(0.6); 2.196 (0.6); 2.178 (0.8); 2.159 (0.6); 1.989 (0.7); 1.980 (0.5); 1.768 (0.4); 1.711 (0.3); 1.628
(0.4); 1.609 (2.5); 1.549 (0.8); 1.491 (0.7); 1.481 (0.8); 1.467 (0.7); 1.423 (0.6); 1.388 (0.7); 1.350
(1.3); 1.298 (1.8); 1.258 (3.2); 1.235 (16.0); 1.165 (2.1); 1.150 (1.4); 0.866 (1.4); 0.854 (2.9); 0.836

(2.1); 0.146 (2.2); 0.008 (16.7); 0.000 (489.3); −0.008 (19.7); −0.150 (2.2)

### USE EXAMPLES

### Example No. 1

Boophilus microplus—Injection Test

Solvent: dimethyl sulfoxide

To produce a suitable preparation of active compound, 10 mg of active compound are mixed with 0.5 ml of solvent and the concentrate is diluted with solvent to the desired concentration.

1 μl of the active compound solution is injected into the abdomen of 5 engorged adult female cattle ticks (Boophilus microplus). The animals are transferred into dishes and kept in a climate-controlled room.

Efficacy is assessed after 7 days by laying of fertile eggs. Eggs which are not visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples show an efficacy of 100% at an application rate of 20 μg/animal: 6, 14

### Example No. 2

Meloidogyne incognita Test

Solvent: 125.0 parts by weight of acetone

To produce a suitable preparation of active compound, 1 part by weight of active compound is mixed with the stated amount of solvent and the concentrate is diluted with water to the desired concentration.

Vessels are filled with sand, active compound solution, an egg/larvae suspension of the southern root-knot nematode (Meloidogyne incognita) and lettuce seeds. The lettuce seeds germinate and the plants develop. The galls develop on the roots.

After 14 days, the nematicidal efficacy in % is determined by the formation of galls. 100% means that no galls were found; 0% means that the number of galls on the treated plants corresponds to the untreated control.

In this test, for example, the following compounds from the preparation examples shows an efficacy of 90% at an application rate of 20 ppm: 17

### Example No. 3

Phaedon cochleariae—Spray Test

| | |
|---|---|
| Solvent: | 78.0 parts by weight of acetone |
| | 1.5 parts by weight of dimethylformamide |
| Emulsifier: | alkylaryl polyglycol ether |

To produce a suitable preparation of active compound, 1 part by weight of active compound is dissolved using the stated parts by weight of solvent and made up with water containing an emulsifier concentration of 1000 ppm until the desired concentration is attained. To produce further test concentrations, the preparation is diluted with emulsifier-containing water.

Discs of Chinese cabbage leaves (Brassica pekinensis) are sprayed with an active compound preparation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples show an efficacy of 100% at an application rate of 500 g/ha: 11

### Example No. 4

Tetranychus urticae—Spray Test, OP-Resistant

| | |
|---|---|
| Solvent: | 78.0 parts by weight of acetone |
| | 1.5 parts by weight of dimethylformamide |
| Emulsifier: | alkylaryl polyglycol ether |

To produce a suitable preparation of active compound, 1 part by weight of active compound is dissolved using the stated parts by weight of solvent and made up with water containing an emulsifier concentration of 1000 ppm until the desired concentration is attained. To produce further test concentrations, the preparation is diluted with emulsifier-containing water.

Discs of bean leaves (Phaseolus vulgaris) infested with all stages of the greenhouse red spider mite (Tetranychus urticae) are sprayed with an active compound preparation of the desired concentration.

After 6 days, the efficacy in % is determined. 100% means that all the spider mites have been killed; 0% means that none of the spider mites have been killed.

In this test, for example, the following compounds from the preparation examples show an efficacy of 100% at an application rate of 500 g/ha: 1, 4, 6, 8, 9, 10, 11, 13, 18

In this test, for example, the following compounds from the preparation examples show an efficacy of 90% at an application rate of 500 g/ha: 2, 5, 7, 14, 17

1. Compound of formula (I) 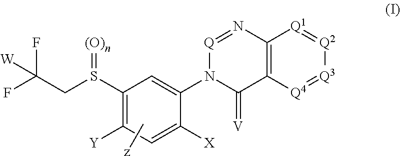
in which
V represents oxygen, sulfur or substituted nitrogen;
Q represents substituted carbon or represents nitrogen;
Q¹, Q², Q³, Q⁴ each independently of one another represent substituted carbon or represent nitrogen, where at least one of the structural elements Q1, Q2, Q3, Q4 is nitrogen and at most two of the structural elements Q1, Q2, Q3, Q4 are nitrogen;
W represents hydrogen or halogen;
n represents the number 0, 1 or 2;
X, Y and z each independently of one another represent hydrogen, halogen, hydroxyl, amino, cyano, nitro, OCN, SCN, SF5; or
represent trialkylsilyl, alkyl, haloalkyl, cyanoalkyl, hydroxyalkyl, alkoxycarbonylalkyl, alkoxyalkyl, alkenyl, haloalkenyl, cyanoalkenyl, alkynyl, haloalkynyl, cyanoalkynyl, alkoxy, haloalkoxy, cyanoalkoxy, hydroxycarbonylalkoxy, alkoxycarbonylalkoxy, alkoxyalkoxy, alkylhydroxyimino, alkoxyimino, alkylalkoxyimino, haloalkylalkoxyimino, alkylthio, haloalkylthio, alkoxyalkylthio, alkylthioalkyl, alkylsulfinyl, halo alkylsulfinyl, alkoxyalkylsulfinyl, alkylsulfinylalkyl, alkylsulfonyl, haloalkylsulfonyl, alkoxyalkylsulfonyl, alkylsulfonylalkyl, alkylsulfonyloxy, alkylcarbonyl, haloalkylcarbonyl, carboxyl, alkylcarbonyloxy, alkoxycarbonyl, haloalkoxycarbonyl, aminocarbonyl, alkylaminocarbonyl, dialkylaminocarbonyl, alkenylaminocarbonyl, dialkenylaminocarbonyl, cycloalkylaminocarbonyl, alkylsulfonylamino, aminosulfonyl, alkylaminosulfonyl, dialkylaminosulfonyl, alkylsulfoximino, aminothiocarbonyl, alkylaminothiocarbonyl or dialkylaminothiocarbonyl, where all the aforementioned radicals may optionally be substituted; or represent phenylalkyl, phenoxy, phenylalkyloxy, phenoxyalkyl, phenylthio, phenylthioalkyl, phenylsulfinyl, phenylsulfonyl, hetarylalkyl, hetaryloxy, hetarylalkyloxy, hetarylthio, hetarylsulfinyl or hetarylsulfonyl, where all the aforementioned radicals may optionally be substituted; or represent cycloalkylalkyl, cycloalkyloxy, cycloalkylalkoxy, cycloalkylthio, cycloalkylalkylthio, cycloalkylsulfinyl, cycloalkylalkylsulfinyl, cycloalkylsulfonyl, cycloalkylalkylsulfonyl or cycloalkenyl, where all the aforementioned radicals may each optionally be substituted; or
represent NR′R″,
where R′ and R″ each independently of one another hydrogen, cyano, alkyl, haloalkyl, cyanoalkyl, hydroxyalkyl, alkoxyalkyl, alkylthioalkyl, alkenyl, haloalkenyl, cyanoalkenyl, alkynyl, haloalkynyl, cyanoalkynyl, acyl or alkoxycarbonyl; or
R′ and R″ together with the nitrogen atom to which they are attached may form an optionally substituted, saturated or unsaturated five- to eight-membered ring optionally interrupted by one or more heteroatoms which are selected independently from the group consisting of O, S and N; or are a 3- to 6-membered saturated, partly saturated or aromatic ring which may optionally contain one to three heteroatoms which are selected independently from the group consisting of O, S and N, and which may optionally be substituted;
or X and z, or Y and z, together with the carbon atoms to which they are attached, form a 5- or 6-membered ring which is optionally substituted and optionally interrupted by one or more heteroatoms which are selected independently from the group consisting of O, S, N and CO.

2. Compound according to claim 1, in which
V represents oxygen;
Q represents C—R or nitrogen;
R represents hydrogen, hydroxy, halogen, cyano, alkyl, cycloalkyl, haloalkyl, alkoxy, haloalkoxy, alkylthio, haloalkylthio, alkylsulfinyl, haloalkylsulfinyl, alkylsulfonyl, haloalkylsulfonyl, amino, monoalkylamino or dialkylamino;
Q¹ represents C—R¹ or nitrogen;
Q² represents C—R² or nitrogen;
Q³ represents C—R³ or nitrogen;
Q⁴ represents C—R⁴ or nitrogen;
where at least one of the structural elements Q¹, Q², Q³, Q⁴ is nitrogen and at most two of the structural elements Q¹, Q², Q³, Q⁴ are nitrogen;
R¹, R², R³, R⁴each independently of one another represent hydrogen, hydroxy, halogen, cyano, alkyl, cycloalkyl, haloalkyl, alkoxy, haloalkoxy, hetaryl, alkylthio, haloalkylthio, alkylsulfinyl, haloalkylsulfinyl, alkylsulfonyl, haloalkylsulfonyl, amino, monoalkylamino or dialkylamino;
W represents hydrogen or halogen;
n represents the number 0 or 1;
X, Y and z independently of one another
represents hydrogen, fluorine, chlorine, bromine, cyano, nitro, amino, hydroxyl, alkyl, haloalkyl, alkenyl, alkynyl, alkoxy, haloalkoxy or aminothiocarbonyl;
or represent benzyl, phenoxy, phenylthio, cyclopropylmethyl, cyclopropyloxy or cyclopropylthio, which is optionally mono- or polysubstituted identically or differently by fluorine, chlorine, bromine, cyano, nitro, hydroxyl, amino, methyl, ethyl, trifluoromethyl, difluoromethyl, trifluoroethyl, difluoroethyl, methoxy, ethoxy, trifluoromethoxy, trifluoroethoxy, or optionally methyl-, fluorine-, chlorine- or cyano-substituted cyclopropyl;
or represent cyclopropyl, cyclobutyl, cyclopentyl, cyclohexyl or phenyl, which is optionally mono- or polysubstituted identically or differently by fluorine, chlorine, bromine, cyano, nitro, hydroxyl, amino, methyl, ethyl, trifluoromethyl, difluoromethyl, trifluoroethyl, difluoroethyl, methoxy, ethoxy, trifluoromethoxy, trifluoroethoxy, or optionally methyl-, fluorine-, chlorine- or cyano-substituted cyclopropyl.

3. Compound according to claim 1, in which
V represents oxygen;
Q represents C—R or nitrogen;
R represents hydrogen, halogen, (C₁-C₄)-alkyl, (C₁-C₄)-haloalkyl, (C₁-C₄)-alkoxy, (C₁-C₄)-haloalkoxy or (C₃-C₆)-cycloalkyl;
Q¹ represents C—R¹ or nitrogen;
Q² represents C—R² or nitrogen;
Q³ represents C—R³ or nitrogen;
Q⁴ represents C—R⁴ or nitrogen;
where at least one of the structural elements Q¹, Q², Q³, Q⁴ is nitrogen and at most two of the structural elements Q¹, Q², Q³, Q⁴ are nitrogen;
R¹, R², R³, R⁴each independently of one another represent hydrogen, halogen, (C₁-C₄)-alkyl, (C₁-C₄)-haloalkyl, (C₁-C₄)-alkoxy, (C₁-C₄)-haloalkoxy or (C₁-C₄)-cycloalkyl;
W represents hydrogen or fluorine;
n represents the number 0 or 1;
X and Y each independently represent hydrogen, fluorine, chlorine, bromine, methyl, ethyl, trifluoromethyl, (2,2)-difluoromethyl, methoxy, difluoromethoxy, trifluoromethoxy, cyclopropyl, cyano, amino, hydroxyl or nitro;
z represents hydrogen.

4. Compound according to claim 1, in which
V represents oxygen;
Q represents C—R or nitrogen;
R represents hydrogen, fluorine, chlorine, methyl, ethyl, tert-butyl, trifluoromethyl, methoxy, trifluoromethoxy or cyclopropyl;
Q¹ represents C—R¹ or nitrogen;
Q² represents C—R² or nitrogen;
Q³ represents C—R³ or nitrogen;
Q⁴ represents C—R⁴ or nitrogen;
where at least one of the structural elements Q¹, Q², Q³, Q⁴ is nitrogen and at most two of the structural elements Q¹, Q², Q³, Q⁴ are nitrogen;
R¹, R², R³, R⁴ each independently of one another represent hydrogen, fluorine, chlorine, methyl, ethyl, tert-butyl, trifluoromethyl, methoxy, trifluoromethoxy or cyclopropyl;
W represents hydrogen or fluorine;
n represents the number 0 or 1;
X represents hydrogen, chlorine, fluorine or methyl;
Y represents chlorine, bromine, cyano, methyl, trifluoromethyl, fluorine or methoxy;
z represents hydrogen.

5. Formulation, optionally suitable for an agrochemical formulation, comprising at least one compound of formula (I) according to claim 1.

6. Formulation according to claim 5, further comprising at least one extender and/or at least one surface-active substance.

7. Formulation according to claim 5, wherein the compound of formula (I) is in a mixture with at least one further active compound.

8. Method for controlling one or more pests, optionally comprising one or more animal pests, comprising allowing a compound of formula (I) according to claim 1 to act on the one or more pests and/or a habitat thereof.

9. Method according to claim 8, wherein the pest is an animal pest and comprises an insect, an acarid or a nematode, or wherein the pest is an insect, an acarid or a nematode.

10. A compound of formula (I) according to claim 1 capable of being used for controlling one or more animal pests.

11. Use A compound according to claim 10, wherein the animal pest comprises an insect, an acarid or a nematode, or wherein the animal pest is an insect, an acarid or a nematode.

12. A compound according to claim 10 capable of being used in crop protection.

13. A compound according to claim 10 capable of being used in the field of animal health.

14. Method for protecting seed or a germinating plant from one or more pests, optionally comprising one or more animal pests, comprising contacting the seed with a compound of formula (I) according to claim 1.

15. Seed obtained by a method according to claim 14.
